(19) United States
(12) Patent Application Publication
(10) Pub. No.: US 2022/0340948 A1
(43) Pub. Date: Oct. 27, 2022

(54) CONCENTRATED PERFUSION MEDIUM

(71) Applicant: Boehringer Ingelheim International GmbH, Ingelheim am Rhein (DE)

(72) Inventors: Jonathan COFFMAN, Gaithersburg, MD (US); Henry LIN, Westfield, NJ (US); Todd LUMAN, Hayward, CA (US); Daisie OGAWA, Oakland, CA (US); Janani RAVIKRISHNAN, Fremont, CA (US); Hayden TESSMAN, San Ramon, CA (US); Samet YILDIRIM, Union City, CA (US); Marcella YU, Fremont, CA (US)

(21) Appl. No.: 17/763,723

(22) PCT Filed: Sep. 25, 2020
(86) PCT No.: PCT/EP2020/076836

§ 371 Date: Mar. 25, 2022

Related U.S. Application Data
(60) Provisional application No. 62/906,983, filed on Sep. 27, 2019.

(51) Int. Cl. C12P 21/00; C12N 15/62; C12N 5/071; C12M 1/00; C12M 1/34

(52) U.S. Cl. CPC C12P 21/005; C12N 15/62; C12N 5/0682; C12M 29/10; C12M 41/36; C12N 2500/90; C12N 2500/60

(57) ABSTRACT

The invention relates to a serum-free cell culture perfusion medium comprising the medium components subgrouped into at least three separate aqueous concentrated feeds and a diluent, wherein the resulting serum-free cell culture perfusion medium is pH-adjusting to neutral pH upon mixing. Also provided is a method of preparing said serum-free cell culture perfusion medium. The invention further relates to methods of culturing mammalian cells or producing a protein of interest in perfusion culture using said serum-free cell culture perfusion medium that achieve high productivity at a low cell specific perfusion rate. The invention further relates to the use of the new and improved serum-free cell culture perfusion medium to control osmolality in a perfusion cell culture, wherein increasing osmolality results in an increase in total productivity and/or cell specific productivity by suppressing cell growth during cell culture, e.g., during production phase of perfusion cell culture. Suppression of cell growth particularly reduces or eliminates the need for wasteful cell bleed.

### TECHNICAL FIELD

The invention relates to a serum-free cell culture perfusion medium comprising the medium components subgrouped into at least three separate aqueous concentrated feeds and a diluent, wherein the serum-free cell culture perfusion medium is pH-adjusting to neutral pH upon mixing. Also provided is a method of preparing said serum-free cell culture perfusion medium. The invention further relates to methods of culturing mammalian cells or producing a protein of interest in perfusion culture using said serum-free cell culture perfusion medium that achieve high productivity at a low cell specific perfusion rate. The invention further relates to the use of the new and improved serum-free cell culture perfusion medium to control osmolality in a perfusion cell culture, wherein increasing osmolality results in an increase in total productivity and/or cell specific productivity by suppressing cell growth during cell culture, e.g., during production phase of perfusion cell culture. Suppression of cell growth particularly reduces or eliminates the need for wasteful cell bleed.

### BACKGROUND

Three methods are typically used in commercial processes for the production of recombinant proteins by mammalian cell culture: batch culture, fed-batch culture, and perfusion culture.

Perfusion based methods offer potential improvements over batch and fed-batch methods, including improved product quality and stability, improved scalability, and increased cell specific productivity. Unlike batch and fed-batch bioreactors, perfusion systems involve the continuous removal of spent media. By continuously removing spent media and replacing it with new media, the levels of nutrients are better maintained which simultaneously optimizes growth conditions and removes cell waste products. The diminished waste products reduce toxicity to the cells and the expression products. Thus, perfusion bioreactors typically result in significantly less protein degradation and thus, a higher quality product. Product can also be harvested and purified much more quickly and continuously, which is particularly effective when producing a product that is unstable.

Perfusion bioreactors are also more easily scalable. As compared to traditional batch or fed-batch systems, perfusion bioreactors offer several advantages with regard to scalability and/or increasing demand. For one, perfusion bioreactors are smaller in size and can produce the same productivity (i.e., product yield) with less volume. It is accepted that perfusion bioreactors can function at 5- to 20-fold concentrations compared to fed-batch bioreactors. For example, a 100-liter perfusion bioreactor can produce the same product yield as a 1,000-liter fed-batch bioreactor. Therefore, the use of a 1,000-liter perfusion bioreactor could conceivably replace a typical 10,000-liter traditional fed-batch bioreactor without negatively impacting the overall productivity. This significant advantage translates into smaller space requirements when expanding production. This may also translate into an array of advantages relating to lower operational utilities, less infrastructure, less labor, reduced complexity of equipment, continuous harvesting, and increased product yields.

Achieving high cell culture densities accounts for part of the greater productivity of perfusion systems. In a typical large scale fed-batch commercial cell culture process, cell densities of 10-50×10⁶ cells/mL can be reached. However, with perfusion-based bioreactors, extreme cell densities of >1×10⁸ cells/mL have been achieved. In addition, in perfusion mode, high cell numbers are sustained for much longer periods of time through the continuous replenishment of spent media. The higher cell densities for increased periods of time in perfusion bioreactors accounts in part for their more efficient performance.

Typical perfusion cultures begin with a batch culture start-up lasting for a day or more to enable rapid initial cell growth and biomass accumulation, followed by continuous, step-wise and/or intermittent addition of fresh perfusion media to the culture and simultaneous removal of spent media with retention of cells throughout the growth and production phases of the culture. Various methods, such as sedimentation, centrifugation, or filtration, can be used to remove spent media, while maintaining the cells. Perfusion flow rates of a fraction of a working volume per day up to many multiple working volumes per day have been utilized.

While continuous perfusion systems have numerous advantages over traditional fed-batch and batch systems, many challenges still remain before perfusion bioreactors become more widely accepted and utilized in the biologics manufacturing industry. For example, perfusion bioreactors consume a significantly greater volume of media than traditional fed-batch systems due to the continuous cycle of removal and replenishment of media. Specifically, the volumes of media required to sustain perfusion rates of 1-3 vessel volumes per day (vvd) become logistically challenging, if not impossible, above the pilot scale (˜100 L bioreactor).

WO 92/22637, formulated concentrated media subgroups separated based on physicochemical properties. However, the concentrated media subgroups are not pH adjusting upon mixing and are hence not suitable for direct addition to the cell culture. Also, the media disclosed in WO 92/22637, such as the minimal media RPMI-1640, DMEM and Ham's F-12, which are not as rich as modern cell culture media, having amino acid concentrations in the mM range rather than the μM range at working concentration.

Another problem facing continuous perfusion cell culture systems is the challenge of maintaining a constant viable cell density, and by consequence, a healthier and more productive cell culture. This has typically been addressed by allowing for “cell bleed.” During cell bleeding, cells are removed and discarded as waste at a rate sufficient to allow for a steady state perfusion cell culture. In turn, this keeps viable cell density constant. A large proportion of the culture medium and hence product can be lost due to the technique of cell bleeding, which siphons off proliferating cells and medium in order to maintain a constant, sustainable viable cell density within the bioreactor. Up to one-third of harvestable material can be lost due to cell bleeding techniques. Using cell bleeding therefore decreases the product yield per run as the product within the portion removed by cell bleeding is not harvested. Therefore, any amount of cell bleeding negatively impacts process efficiency, product recovery and most importantly results in product loss. The cell bleed rate is determined by rate of cell growth. A faster doubling time also necessitates a higher cell bleed to maintain constant cell density, and consequently, more waste.

As an alternative to cell bleeding, others have tried chemical additives to slow the rate of cell growth. Reduced cell growth typically also increases cell specific productivity. For example, Du et al. (Biotechnology and Bioengineering, Vol. 112, No. 1, January 2015) reported the use of a small molecule cell cycle inhibitor to control growth and improve cell culture productivity. A similar disclosure is found in WO 2014/109858, which discloses the use of CDK4 inhibitor in cell culture such as batch, fed-batch and perfusion culture. Du et al. further teaches that CDK4/6 inhibitors specifically inhibit the cell cycle without affecting other cellular targets. However, the addition of inhibitors and compounds not required for cell growth and/or cell maintenance are to be avoided. Thus, additional methods that are effective to suppress cell growth in the perfusion state and avoid the need for cell bleeding would significantly help advance the art.

Osmolality has been a known lever to impact cell growth. Prior art using osmolality to affect cell growth is known in the literature (Zhu, et al (2005) Biotechnology Progress 21, 70-77; Han, Koo and Lee (2009) Biotechnology Progress 25, 1440-1447; Hu and Aunins (1997) Current Opinion in Biotechnology, 148-153). However, the ability to control a cell culture process to a target osmolality, particularly in perfusion culture has never been established. Also, chemical additives affect media composition and/or need to be cleared in subsequent purification steps, thereby increasing process complexity. Chemical additives including salts may also affect product quality.

In view of the challenges with perfusion cell culture such as prepared media consumption and the desire to further increased productivity, media that improve logistic problems and methods that are effective in suppressing cell growth in the perfusion state and avoid the need for cell bleeding without further additives would significantly help advance the art.

### SUMMARY OF THE INVENTION

The present invention relates in part to the discovery that feed media can be developed in a more concentrated form by compartmentalization to reduce the cell specific perfusion rate and the volume of prepared media consumed. These concentrated feeds are diluted in the bioreactor vessel. It is advantageously used with sterilized, de-ionized water as a diluent which does not require preparation other than filtering. Additionally, the unique combination of the 3 media concentrates (acidic, basic, and near neutral) designed in this invention allows for the use of higher-fold concentrates, thereby reducing the total media volume further. By reducing the prepared volume of media, a perfusion process has been developed that takes away media volume as a bottleneck, therefore can justify scale up of the perfusion cell culture process to 1000 L scale and potentially above.

The use of separate concentrated feeds and a diluent also allows to control cell growth by culture osmolality. Using the concept of mass balance, an osmo balance was derived with known osmolality of each of the concentrated feeds, feed rates, calculated daily cell specific osmolality consumption rate, to predict the culture residual osmolality as an output. Using this method, the growth of the cell culture can be controlled by increasing the residual osmolality to physiologically stressful levels (about 350-400 mOsm or higher) while remaining below the cytotoxic level (about 400 mOsm or higher). The physiologically stressful levels as well as the cytotoxic level may be cell line specific. However, this can be easily determined by measuring the viable cell concentrations and viability at different osmolality levels during cultivation, which may be performed in a small scale such as 3 ml working volume. In the current invention, the culture osmolality is controlled via an osmo balance which includes changes in feed rates of the media concentrates from a day-to-day basis at a fixed vessel volume per day (VVD) or changes in feed rates of the WD from a day-to day basis at fixed feed rates of the media concentrates. The osmo balance is capable of targeting a higher or lower residual osmolality by adjusting concentrates and diluent rate, whereas the chemical additives approach that others have used can only adjust osmolality in the increasing direction. It was found that the osmo balance as described herein was effective in suppressing cell growth, and that this growth suppression led to an increase in cell specific productivity and helped in maintaining high viability in a cell culture.

The cell growth suppression by the osmo balance described herein not only leads to an increase in cell specific productivity and sustained high cell viability, in perfusion cell culture it also reduces or eliminates the need to employ cell bleeding techniques during the perfusion state to otherwise maintain the cells in a steady state of growth. This reduces or eliminates product loss due to wasteful and undesirable cell bleeding techniques.

The tripartite highly concentrated feed media provided herein can theoretically be used in connection with any type of cell culture system, but are particularly advantageous in continuous perfusion cell culture systems. Thus the serum-free cell culture perfusion medium according to the invention is particularly suitable for use in a continuous perfusion cell culture system. Also the osmo balance may theoretically be used in connection with any type of cell culture system. However, it is particularly advantageous when the cell culture system is a continuous perfusion cell culture system.

In one aspect the invention relates to a compartmentalized serum-free cell culture perfusion medium comprising the medium components subgrouped into at least three separate aqueous concentrated feeds and a diluent, wherein the first concentrated feed is an alkaline concentrated feed, the second concentrated feed is an acidic concentrated feed and the third concentrated feed is a near neutral concentrated feed; wherein the compartmentalized serum-free cell culture perfusion medium is pH-adjusting to neutral pH upon mixing of the at least three separate aqueous concentrated feeds and the diluent in the resulting serum-free cell culture perfusion medium. In a preferred embodiment the at least three separate aqueous concentrated feeds are not premixed prior to addition to the cell culture and/or the reaction vessel of the bioreactor. The diluent is preferably sterile water. In one embodiment the resulting serum-free cell culture perfusion medium has a pH of between 6.7 and 7.5, between 6.9 and 7.4, preferably between 6.9 and 7.2, upon mixing of the at least three separate aqueous concentrated feeds and the diluent. The compartmentalized serum-free cell culture perfusion medium according to the invention is suitable for separate addition of the alkaline concentrated feed, the acidic concentrated feed and the near neutral concentrated feed to a cell culture and/or a reaction vessel of a bioreactor; direct addition of the alkaline concentrated feed, the acidic concentrated feed and the near neutral concentrated feed to a cell culture and/or a reaction vessel of a bioreactor without prior pre-mixing; and/or direct mixing of the at least three separate aqueous concentrated feeds in a cell culture and/or a reaction vessel of a bioreactor.

In certain embodiments, the alkaline concentrated feed is a 2× to 80× concentrated feed, the acidic concentrated feed is a 2× to 40× concentrated feed and the near neutral concentrated feed is a 2× to 50× concentrated feed. The near neutral concentrated feed has a pH of about 6.5 to about 8.5. Preferably, the alkaline concentrated feed has a pH of about 9 or higher, the acidic concentrated feed has a pH of about 5 or lower and the near neutral concentrated feed has a pH of about 7 to about 8.5. Also, the ratio (v/v/v) of the alkaline concentrated feed to the acidic concentrated feed to the near neutral concentrated feed is a fixed ratio to provide the resulting serum-free cell culture perfusion medium that is pH-adjusting to a neutral pH; and the ratio (v/v) of the diluent to the cumulative volume of the at least three separate aqueous concentrated feeds in the resulting serum-free cell culture perfusion medium that is pH-adjusting to a neutral pH determines the osmolality of the serum-free cell culture perfusion medium.

The acidic concentrated feed may comprise trace elements, trace metals, inorganic salts, chelators, polyamines, and regulatory hormones. The acidic concentrated feed and/or the near neutral concentrated feed may comprise surfactants, anti-oxidants, and carbon sources. Further, the alkaline concentrated feed comprises amino acids with maximum solubility at alkaline pH of 9 or higher, preferably comprising at least aspartic acid, histidine and tyrosine, and optionally cysteine and/or cystine and/or folic acid. The remaining amino acids are in the acidic and/or near neutral concentrated feed, preferably in the acidic concentrated feed. Preferably the vitamins and the metals are in separate feeds, preferably vitamins are in the near neutral feed and metals are in the acidic feed. Vitamins poorly soluble in aqueous solutions, such as choline chloride, are present in the neutral feed and the acidic feed.

The invention also relates to an alkaline aqueous concentrated feed for combination with an acidic aqueous concentrated feed, a near neutral aqueous concentrated feed and a diluent to form a serum-free cell culture perfusion medium, wherein the pH of the serum-free cell culture perfusion medium is automatically adjusted to a neutral pH. In another embodiment the invention relates to an acidic aqueous concentrated feed for combination with an alkaline aqueous concentrated feed, a near neutral aqueous concentrated feed and a diluent to form a serum-free cell culture perfusion medium, wherein the pH of the resulting serum-free cell culture perfusion medium is automatically adjusted to a neutral pH. In yet another aspect the invention relates to a near neutral aqueous concentrated feed for combination with an alkaline aqueous concentrated feed, an acidic aqueous concentrated feed and a diluent to form a serum-free cell culture perfusion medium, wherein the pH of the resulting serum-free cell culture perfusion medium is automatically adjusted to a neutral pH.

In yet another aspect the invention relates to a method of preparing a serum-free cell culture perfusion medium, the method comprising (a) providing the components of a cell culture media in at least three subgroups of components based on solubility at alkaline, acidic and neutral pH, (b) dissolving (i) the subgroup of components soluble at alkaline pH in an alkaline aqueous solution to form an alkaline concentrated feed; (ii) the subgroup of components soluble at acidic pH in an acidic aqueous solution to form an acidic concentrated feed; and (iii) the subgroup of components soluble at neutral pH in a neutral aqueous solution to form a near neutral concentrated feed; (c) optionally storing the prepared alkaline concentrated feed, acidic concentrated feed and near neutral concentrated feed in separate containers; and (d) adding the prepared alkaline concentrated feed, acidic concentrated feed and near neutral concentrated feed and the diluent to the cell culture and/or the reaction vessel of the bioreactor, wherein (i) the alkaline concentrated feed, the acidic concentrated feed and the near neutral concentrated feed are added separately to the cell culture and/or the reaction vessel of the bioreactor; and (ii) the diluent is added separately to the cell culture and/or the reaction vessel of the bioreactor or the diluent is premixed with one of the at least three separate aqueous concentrated feeds immediately before addition to the cell culture and/or the reaction vessel of the bioreactor; wherein the pH of the resulting serum-free cell culture perfusion medium is automatically pH adjusted to an about neutral pH upon mixing of the at least three separate aqueous concentrated feeds and the diluent. The diluent is preferably sterile water. In one embodiment the resulting serum-free cell culture perfusion medium prepared by the method has a pH of between 6.7 and 7.5, between 6.9 and 7.4, preferably between 6.9 and 7.2, upon mixing of the at least three separate aqueous concentrated feeds and the diluent.

In certain embodiments the at least three concentrated feeds are added drop-wise through separate ports to the cell culture and/or the reaction vessel of the bioreactor. The in-vessel mixing and dilution of the at least three separate aqueous concentrated feeds allows 50-90%, preferably 60-90% lower prepared medium consumption over a culture period of 14 days compared to a serum-free cell culture perfusion medium mixed and diluted prior to addition to the bioreactor. Typically the cell culture and/or the reaction vessel of the bioreactor comprise(s) mammalian cells. Also the method further comprises a step of sterilizing the concentrated feeds prior to storage and/or addition to the cell culture and/or the reaction vessel of the bioreactor.

In certain embodiments the alkaline concentrated feed is a 2× to 80× concentrated feed, wherein the acidic concentrated feed is a 2× to 40× concentrated feed and the near neutral concentrated feed is a 2× to 50× concentrated feed. The near neutral concentrated feed has a pH of 6.5-8.5. Preferably, the alkaline concentrated feed has a pH of 9 or higher, the acidic concentrated feed has a pH of 5 or lower and the near neutral concentrated feed has a pH of 7 to 8.5. Also, the ratio (v/v/v) of the alkaline concentrated feed to the acidic concentrated feed to the near neutral concentrated feed is a fixed ratio to provide the resulting serum-free cell culture perfusion medium that is pH-adjusting to a neutral pH in the cell culture and/or the reaction vessel of the bioreactor; and the ratio (v/v) of the diluent to the cumulative volume of the at least three separate aqueous concentrated feeds added to the cell culture and/or the reaction vessel of the bioreactor to provide the resulting serum-free cell culture perfusion medium that is pH-adjusting to a near neutral pH determines the osmolality of the serum-free cell culture perfusion medium in the cell culture and/or the reaction vessel of the bioreactor. In certain embodiment, the cell culture and/or the reaction vessel of the bioreactor comprise at least about 100 L serum-free cell culture perfusion medium, preferably at least about 1000 L serum-free cell culture perfusion medium. Preferably the cell culture has a volume of at least about 100 L and/or the bioreactor has a volume of at least about 100 L. More preferably the cell culture has a volume of at least about 1000 L and/or the bioreactor has a volume of at least about 1000 L.

Also provided is a serum-free cell culture perfusion medium obtainable by the method according to the invention.

In another aspect the invention relates to a method of culturing mammalian cells expressing a heterologous protein in perfusion culture, comprising: (a) inoculating a bioreactor with mammalian cells expressing a heterologous protein in a serum-free cell culture medium; (b) culturing the mammalian cells in a perfusion culture by continuously feeding the mammalian cells with a serum-free cell culture perfusion medium feed and removing spent media while keeping the cells in culture, wherein the serum-free cell culture perfusion medium feed is (i) a compartmentalized serum-free cell culture perfusion medium comprising the medium components subgrouped into at least three separate aqueous concentrated feeds and a diluent, wherein the first concentrated feed is an alkaline concentrated feed, the second concentrated feed is an acidic concentrated feed and the third concentrated feed is a near neutral concentrated feed; and wherein the compartmentalized serum-free cell culture perfusion medium is pH-adjusting to neutral pH upon mixing of the at least three separate aqueous concentrated feeds and the diluent in the resulting serum-free cell culture perfusion medium; and/or (ii) the serum-free cell culture perfusion medium obtainable by the method according to the invention, and wherein the alkaline concentrated feed, the acidic concentrated feed and the near neutral concentrated feed of the serum-free cell culture perfusion medium feed are added separately to the cell culture and/or the reaction vessel of the bioreactor and wherein the diluent is added separately to the cell culture and/or the reaction vessel of the bioreactor or the diluent is premixed with one of the at least three separate aqueous concentrated feeds immediately before addition to the cell culture and/or the reaction vessel of the bioreactor. The method typically further comprises harvesting the heterologous protein from the cell culture.

The mammalian cells may initially be cultured as a batch culture before perfusion culture is started and/or perfusion culture starts from days 0 to day 3 of the culture, i.e., post-inoculation. Typically the perfusion rate increases after perfusion has started until a target viable cell density has been reached. In certain embodiments the perfusion rate increases from less or equal to 0.5 vessel volumes per day to about 5 vessel volumes per day, or from less or equal to 0.5 vessel volumes per day to about 2 vessel volumes per day.

In certain embodiments the osmolality of the serum-free cell culture perfusion medium is increased above the optimal osmolality level for growth, resulting in growth suppression at a target viable cell density, preferably wherein the osmolality level of the serum-free cell culture perfusion medium is increased gradually or stepwise starting at about half the target viable cell density. The target viable cell density is about 30×10⁶ cells/ml or higher, about 60×10⁶ cells/ml or higher, about 80×10⁶ cells/ml, preferably about 100×10⁶ cells/ml or higher. Osmolality may be controlled using (a) a constant concentrated feed perfusion rate and a varying diluent perfusion rate, resulting in a varying overall perfusion rate; or (b) a constant overall perfusion rate and a varying concentrated feed perfusion rate; wherein the at least three concentrated feeds are added at a fixed ratio (v/v/v) to each other depending on their fold-concentration to maintain the relative proportion of the medium components in the 1× serum-free cell culture perfusion medium. Thus, the osmolality may be increased using (a) a constant concentrated feed perfusion rate and a decreased diluent perfusion rate, resulting in a decreased overall perfusion rate; or (b) a constant overall perfusion rate and an increased concentrated feed perfusion rate with a decreased diluent perfusion rate; wherein the at least three concentrated feeds are added at a fixed ratio (v/v/v) to each other depending on their fold-concentration to maintain the relative proportion of the medium components in the 1× serum-free cell culture perfusion medium. Preferably no further additive is added to the culture for increasing the osmolality.

The person skilled in the art will know how to determine the optimal osmolality level for growth of the mammalian cell. In one embodiment the optimal osmolality level for growth of the mammalian cell is about 280 to less than 350 mOsm. The osmolality is maintained at a level optimal for growth until about half the target viable cell density is reached. Preferably the osmolality is increased gradually or stepwise starting at about half the target viable cell density, preferably to about 10-50% of the optimal osmolality level for growth. The osmolality is increased to and maintained at an osmolality level that suppresses cell growth at about the target viable cell density, wherein the osmolality level that suppresses cell growth of the mammalian cell in one embodiment is about 350 mOsm or higher, preferably about 380 mOsm or higher. Increasing the osmolality reduces or eliminates the need for cell bleeding during production phase.

When increasing osmolality, cell growth is suppressed to maintain a sustainable viable cell density without cell bleeding. The yield of the heterologous protein produced in the cell culture is increased by at least 5-50% relative to the yield in a control cell culture, wherein the osmolality is not increased.

Generally using the method of the present invention the cell specific perfusion rate (pl/cell/day) is reduced by at least 50% relative to the cell specific perfusion rate of a 1× serum-free cell culture medium.

In certain embodiment, the cell culture and/or the reaction vessel of the bioreactor comprise at least about 100 L serum-free cell culture perfusion medium, preferably at least about 1000 L serum-free cell culture perfusion medium. Preferably the cell culture has a volume of at least about 100 L and/or the bioreactor has a volume of at least about 100 L. More preferably the cell culture has a volume of at least about 1000 L and/or the bioreactor has a volume of at least about 1000 L.

The heterologous protein may be a therapeutic protein, an antibody, or a therapeutically effective fragment thereof. The mammalian cell may be any cell line, such as selected from the group consisting of Chinese Hamster Ovary (CHO) cells, Jurkat cells, 293 cells, HeLa cells, CV-1 cells, or 3T3 cells, or a derivative of any of these cells. The CHO cell can be further selected from the group consisting of a CHO-DG44 cell, a CHO-K1 cell, a CHO DXB11 cell, a CHO-S cell, and a CHO GS deficient cell or a mutant thereof.

According to the method according to the invention further one or more supplements selected from the list of anti-foaming agents, base, glutamine and glucose may be added separately (i.e., additionally) to the cell culture.

Also provided is a method of producing a therapeutic protein using the methods according to the invention.

Also provided is a use of the compartmentalized serum-free cell culture perfusion medium according to the invention or the serum-free cell culture perfusion medium obtainable by the method according to the invention for culturing mammalian cells, particularly for culturing mammalian cells in a perfusion culture. In certain embodiments, the cell specific perfusion rate (pl/cell/day) is reduced by at least 30% relative to the cell specific perfusion rate of a 1× serum-free cell culture medium. Also provided is a use of the compartmentalized serum-free cell culture perfusion medium according to the invention for separate addition of the at least three separate aqueous concentrated feeds to a cell culture and/or a reaction vessel of a bioreactor.

Furthermore the invention provides using the compartmentalized serum-free cell culture perfusion medium according to the invention or the serum-free cell culture perfusion medium obtainable by the method according to the invention for controlling osmolality in a perfusion cell culture. Increasing the osmolality in the cell culture suppresses cell growth and increases heterologous protein production. The yield of the heterologous protein produced in the cell culture is increased by at least 5-50% relative to the yield in a control cell culture, wherein the osmolality is not increased. In one embodiment growth suppression is sufficient to maintain a sustainable viable cell density without cell bleeding.

### BRIEF DESCRIPTION OF THE DRAWINGS

FIG. 1. Bioreactor and feed set-up illustrating separate inlet additions of: acidic, basic, and neutral feeds, and diluent.

FIG. 2. Reactor volume exchanges or Perfusion rate over time, given in liter of the media (_{Lmedia}) per liter of the bioreactor (L_{br}) and day, for an example of a typical operating perfusion rate for perfusion cultures with a feeding strategy using the combination of three media concentrates: the combination of the three media concentrates (MCs; lower dashed line), MCs combined with diluent (solid line), and a potential maximum perfusion rate of the combine feeds (upper dashed line; VVD means “vessel volumes per day”).

FIG. 3. Viable cell density (+/−3 standard deviations; solid lines) and viability (+/−3 SD; dashed lines) for three 100 L bioreactor runs using the concentrated media feed+diluent feeding scheme. Inherent peak VCD (that is, without high osmo inhibition of growth) for this cell line is >200e6 c/mL. By increasing osmolality pre-peak, the culture growth is inhibited and peak VCD suppressed.

FIG. 4. Osmolality (mOsm) of three 100 L bioreactor runs showing increasing osmolality until approximately day 6, when target viable cell density was reached. From peak VCD, the osmolality is held at >380 mOsm to suppress cell proliferation.

FIG. 5. Rector volume exchanged (aka perfusion rate) for three 100 L bioreactor runs using concentrated media feeds fixed at 0.5 vessel volumes per day (VVD), with varying diluent volume. Varying diluent volume controlled residual osmolality in the culture vessel.

FIG. 6. Permeate productivity (g/L_{bioreactor}/day) for three 100 L bioreactors using concentrated media feeds fixed at 0.5 vessel volumes per day (VVD) with varying diluent volume (overall perfusion rate varies). Permeate productivity is calculated by the daily instantaneous titer of the permeate (g/L_{media}), as measured by the Cedex BioAnalyzer, multiplied by the daily perfusion rate (L_{media}/L_{bioreactor}/day).

FIG. 7. Daily specific productivity (Qp, pg/cell/day) of CHO cell culture expressing a recombinant IgG for three 100 L bioreactors using concentrated media feeds fixed at 0.5 vessel volumes per day (VVD) with varying diluent volume (overall perfusion rate varies). Daily Qp is approximated by summing the total productivity of the bioreactor system (that is, the product recovered through the permeate and the product retained within the bioreactor) and dividing by the daily viable cell density (VCD).

FIG. 8. Cell-specific perfusion rate (nL/cell/day) for CHO cells in three 100 L bioreactors using concentrated media feeds fixed at 0.5 vessel volumes per day (VVD) with varying diluent volume (overall perfusion rate varies).

FIG. 9. Viable cell density (VCD, e5 c/mL; solid lines) and Viability (%; dashed lines) for three BI CHO cell lines A (⋄), B (□), and C (Δ) expressing different recombinant IgG molecules. Data are from 2 L bioreactor scale using three concentrated media feeds and sterile water diluent in varying proportions to maintain target residual osmolality, at a constant perfusion rate of two vessel volumes per day (VVD).

FIG. 10. Residual culture osmolality (mOsm) for three BI CHO cell lines A (⋄), B (□), and C (Δ) expressing different recombinant IgG molecules. Data are from 2 L bioreactor scale using three concentrated media feeds and sterile water diluent in varying proportions to maintain target residual osmolality, at a constant perfusion rate of two vessel volumes per day (VVD).

FIG. 11. Reactor volume exchanged (aka perfusion rate; L media/L bioreactor/day) for three BI CHO cell lines A (⋄), B (□), and C (Δ) expressing different recombinant IgG molecules. Data are from 2 L bioreactor scale using three concentrated media feeds and sterile water diluent in varying proportions to maintain target residual osmolality, at a constant perfusion rate of two vessel volumes per day (VVD).

FIG. 12. Permeate productivity (g/L_{bioreactor}/day) for three BI CHO cell lines A (⋄), B (□), and C (Δ) expressing different recombinant IgG molecules. Permeate productivity is calculated by the daily instantaneous titer of the permeate (g/L), as measured by the Cedex BioAnalyzer, multiplied by the daily perfusion rate (L_{media}/L_{bioreactor}/day). Data are from 2 L bioreactor scale using three concentrated media feeds and sterile water diluent in varying proportions to maintain target residual osmolality, at a constant perfusion rate of two vessel volumes per day (VVD).

FIG. 13. Daily specific productivity (Qp, pg/cell/day) for three BI CHO cell lines A (⋄), B (□), and C (Δ) expressing different recombinant IgG molecules. Daily Qp is approximated by summing the total productivity of the bioreactor system (that is, the product recovered through the permeate and the product retained within the bioreactor) and dividing by the daily viable cell density (VCD). Data are from 2 L bioreactor scale using three concentrated media feeds and sterile water diluent in varying proportions to maintain target residual osmolality at a constant perfusion rate of two vessel volumes per day (VVD).

FIG. 14. Cell-specific perfusion rate (CSPR; nL/cell/day) for three BI CHO cell lines A (⋄), B (□), and C (Δ) expressing different recombinant IgG molecules. Data are from 2 L bioreactor scale using three concentrated media feeds and sterile water diluent in varying proportions to maintain target residual osmolality at a constant perfusion rate of approximately two vessel volumes per day (VVD). Variation in CSPR between cell lines is due to differences in viable cell densities (VCD) (see FIG. 9 for VCD and viability). Proportion of feeds relative to each other is kept constant while the overall rate of feeds to diluent is adjusted according to a mass balance of osmolality calculation following the equation: Osmo input=Osmo output+osmo consumption, where osmo input is the osmolality of media concentrate feeds and diluent perfusing into the bioreactor, osmo output is the residual osmolality of the bioreactor supernatant, and osmo consumption is the difference in osmolality between the input and output. The osmo consumption is used to calculate the necessary osmo input for a given desired osmo output. The respective concentrated feeds and diluent perfusion rates are then calculated to achieve the necessary osmo input at an overall perfusion rate of 2 vvd.

FIG. 15. A CHO DG44 cell line expressed in the dihydrofolate reductase (dhfr) selection system (cell line A, Δ) and two different CHO-K1 cell lines run in duplicates (cell line B □, ⋄; cell line C x, x) expressed in the glutamine synthetase (GS) selection system were cultured in a 2 L bioreactor using three concentrated media feeds fixed at a total of 0.5 vessel volumes per day (VVD) with varying diluent volume. All cell lines express a different recombinant IgG molecule. Shown is (A) viable cell densities (VCD; e5 c/mL); (B) viability (%); (C) permeate productivity (g/L/day), with the permeate productivity being calculated from the daily instantaneous titer of the permeate (g/L_{media}), as measured by the Cedex BioAnalyzer, multiplied by the daily perfusion rate (L_{media}/L_{bioreactor}/day), and (D) perfusion rate expressed in reactor volume exchange (L_{media}/L_{bioreactor}/day).

FIG. 16. A CHO-K1 cell line expressing a recombinant IgG in the glutamine synthetase (GS) selection system were cultured in 2 L bioreactors. Runs were performed in either the “MCs vary, total VVD fixed” (⋄) or “MCs fixed, total VVD vary” (□) perfusion control modes. “MCs vary, total WD fixed” refers to a constant total vessel volume per day (VVD) perfusion rate achieved by varying the perfusion rate of the combined Media Concentrates (MCs) and concomitantly varying diluent rate to maintain 2 WD. “MCs fixed, total WD vary” refers to a constant perfusion rate of MCs at 0.5 VVD with a varying diluent perfusion rate, for an overall fluctuating perfusion rate. Shown is (A) viable cell density (VCD, e5 c/mL; primary axis) and viability (%; secondary axis), (B) osmolality (mOsm), (C) Adjusted productivity (g/L_{bioreactor}/d) and (D) reactor volume exchange (L_{media}/L_{bioreactor}/day).

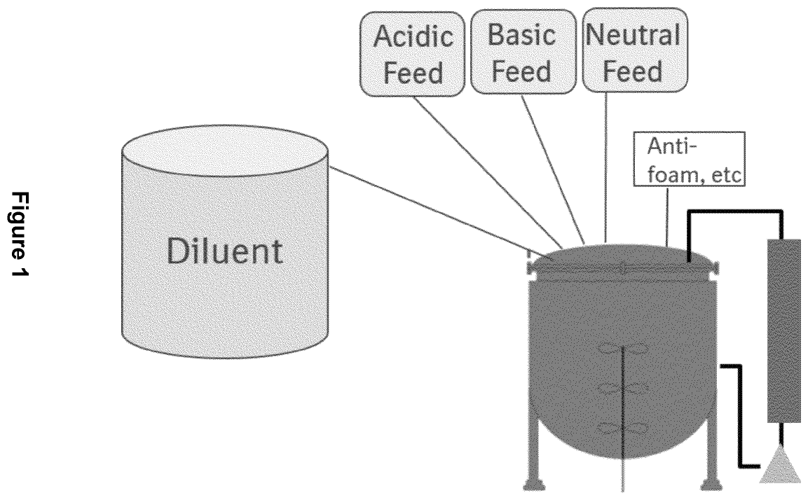

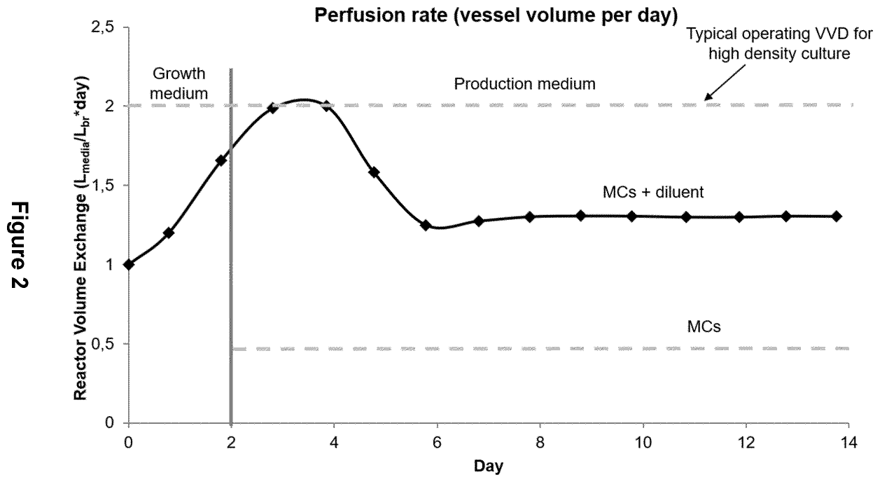

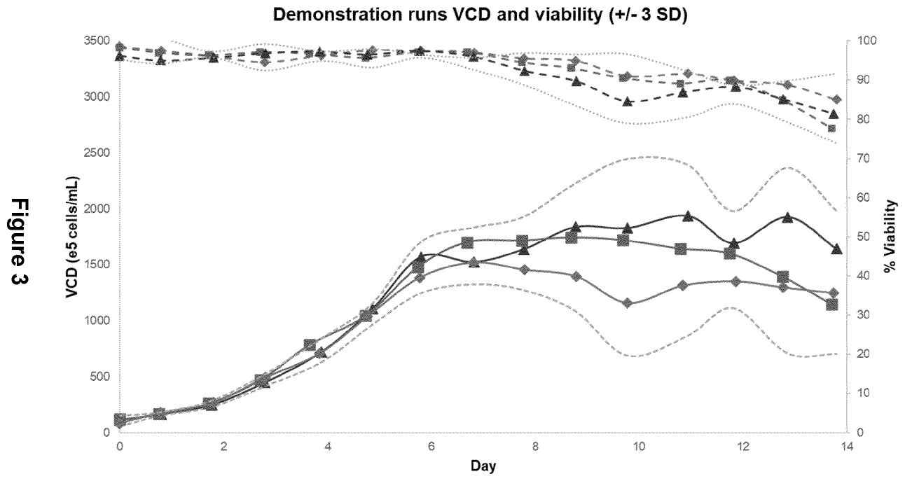

### DETAILED DESCRIPTION

Definitions of certain terms are provided below. In general, any terms presented in this disclosure should be given their ordinary meaning in the art, unless otherwise stated or defined.

The general embodiments “comprising” or “comprised” encompass the more specific embodiment “consisting of”. Furthermore, singular and plural forms are not used in a limiting way. As used herein, the singular forms “a”, “an” and “the” designate both the singular and the plural, unless expressly stated to designate the singular only.

The term “perfusion” as used herein refers to maintaining a cell culture bioreactor in which equivalent volumes of media are simultaneously added and removed from the reactor while the cells are retained in the reactor. A perfusion culture may also be referred to as continuous culture. This provides a steady source of fresh nutrients and constant removal of cell waste products. Perfusion is commonly used to attain much higher cell density and thus a higher volumetric productivity than conventional bioreactor batch or fed batch conditions. Secreted protein products can be continuously harvested while retaining the cells in the reactor, e.g., by filtration, alternating tangential flow (ATF), cell sedimentation, ultrasonic separation, hydrocyclones, or any other method known to the person skilled in the art or as described Kompala and Ozturk (Cell Culture Technology for Pharmaceutical and Cell-Based Therapies, (2006), Taylor & Francis Group, LLC, pages 387-416). Mammalian cells may be grown in suspension cultures (homogeneous cultures) or attached to surfaces or entrapped in different devices (heterogeneous cultures). In order to keep the working volume in the bioreactor constant the harvest rate and cell bleed (fluid removal) should be equal to the predetermined perfusion rate. The culture is typically initiated by a batch culture and the perfusion is started on day 2-3 after inoculation when the cells are still in exponential growth phase and before nutrient limitation occurs. Inoculation at high seeding density (5×10⁶ cells/ml or higher) may necessitate an earlier or even immediate start of perfusion. Thus, perfusion may be started from day 0 to day 4 post-inoculation, preferably from day 0 to day 3 post-inoculation.

Perfusion based methods offer potential improvement over the batch and fed-batch methods by adding fresh media and simultaneously removing spent media. Large scale commercial cell culture strategies may reach high cell densities of 60-90×10⁶ cells/mL, at which point about a third to over half of the reactor volume may be biomass. With perfusion based culture, extreme cell densities of >1×10⁸ cells/mL have been achieved. Typical perfusion cultures begin with a batch culture start-up lasting for a day or more to enable rapid initial cell growth and biomass accumulation, followed by continuous, step-wise and/or intermittent addition of fresh feed media to the culture and simultaneous removal of spent media with retention of cells throughout the growth and production phases of the culture. Various methods, such as sedimentation, centrifugation, or filtration, can be used to remove spent media, while maintaining the cells. Perfusion flow rates of a fraction of a vessel volume per day (VVD) up to many multiple vessel volumes per day have been utilized.

The term “perfusion rate” as used herein is the volume added and removed and is typically measured per day. It depends on the cell density and the medium. It should be minimized to reduce the dilution of the product of interest, i.e., harvest titer, while ensuring adequate rates of nutrient addition and by-product removal. Perfusion is typically started on day 0-3 after inoculation when the cells are still in the exponential growth phase and hence perfusion rate may be increased over the culture. Increase in perfusion rate may be incremental or continuously, i.e., based on cell density or nutrient consumption. It typically starts with 0.5 or 1 vessel volume per day (VVD) and may go up to about 5 WD. Preferably, the perfusion rate is between 0.5 to 2 WD. The increase may be by 0.5 to 1 WD per day. For continuous increase in perfusion, a biomass probe may be interfaced with the harvest pump, such that the perfusion rate is increased as a linear function of the cell density determined by the biomass probe, based on a desired cell specific perfusion rate (CSPR). The CSPR equals the perfusion rate per cell density and an ideal CSPR depend on the cell line and the cell medium. The ideal CSPR should result in optimal growth rate and productivity. A CSPR of 50 to 100 pL/cell per day may be a reasonable starting range, which can be adjusted to find the optimal rate for a specific cell line. Using the at least three separate aqueous concentrated feeds, the supply of nutrients is decoupled from the overall VVD and CSPR allowing a very low CSPR, such as 5 to 20 pL/cell/day preferably even 5 to 10 pL/cell/day. This considerably reduces medium consumption and particularly prepared medium consumption, such as over a culture period of 14 days compared to a serum-free cell culture perfusion medium mixed and diluted prior to addition to the bioreactor or compared to a conventional 1× serum-free cell culture perfusion medium.

The term “steady state” as used herein refers to the condition where cell density and bioreactor environment remain relatively constant. This can be achieved by cell bleeding, nutrient limitation and/or temperature reduction. In most perfusion cultures nutrient supply and waste removal will allow for constant cell growth and productivity and cell bleeding is required to maintain a constant viable cell density or to maintain the cells in steady state. A typical viable cell density at steady state is 10 to 50×10⁶ cells/ml. The viable cell density may vary depending on the perfusion rate. A higher cell density can be reached by increasing the perfusion rate or by optimizing the medium for use with perfusion. At a very high viable cell density perfusion cultures become difficult to control within a bioreactor.

The terms “cell bleed” and “cell bleeding” are used interchangeably herein and refer to the removal of cells and medium from the bioreactor in order to maintain a constant, sustainable viable cell density within the bioreactor. The constant, sustainable viable cell density may also be referred to as target cell density. This cell bleed may be done using a dip tube and a peristaltic pump at a defined flow rate. The tubing should have the right size with a too narrow tube being prone to cell aggregation and clogging while if too large the cells may settle. The cell bleed can be determined based on growth rate, thus viable cell density can be limited to a desired volume in a continuous manner. Alternatively, cells may be removed at a certain frequency, e.g., once a day, and replaced by media to maintain cell density within a predictable range. Ideally the cell bleed rate is equal to the growth rate to maintain a steady cell density.

Typically the product of interest removed with the cell bleed is discarded and therefore lost for the harvest. Opposite to a permeate, the cell bleed contains cells, which makes storage of the product prior to purification more difficult and can have detrimental effects on product quality. Thus, the cells would have to be removed continuously prior to storage and product purification, which would be laborious and cost inefficient. For slow growing cells the cell bleed may be about 10% of the removed fluid and for fast growing cells the cell bleed may be about 30% of the removed fluid. Thus, the product loss through the cell bleed may be about 30% of the product produced in total. The “permeate” as used herein refers to the harvest from which the cells have been separated to be retained in the culture vessel.

The term “culture” or “cell culture” is used interchangeably and refer to a cell population that is maintained in a medium under conditions suitable to allow survival and/or growth of the cell population. The present invention only relates to mammalian cell cultures, and particularly to mammalian perfusion cell cultures. Mammalian cells may be cultured in suspension or while attached to a solid support. As will be clear to the person skilled in the art a cell culture refers to a combination comprising the cell population and the medium in which the population is suspended. The particular type of cell culture is not particularly limited and may encompass all forms and techniques of cell culture, including but not limited to, perfusion, continuous, finite, suspension, adherent or monolayer, anchorage-dependent, and 3D cultures. As used herein the term cell culture refers to a serum-free cell culture

The term “culturing” as used herein refers to a process by which mammalian cells are grown or maintained under controlled conditions and under conditions that supports growth and/or survival of the cells. The term “maintaining cells” as used herein is used interchangeably with “culturing cells”. Culturing may also refer to a step of inoculating cells in a culture medium.

As used herein, the term “batch culture” is a discontinuous method where cells are grown in a fixed volume of culture media for a short period of time followed by a full harvest. Cultures grown using the batch method experience an increase in cell density until a maximum cell density is reached, followed by a decline in viable cell density as the media components are consumed and levels of metabolic by-products (such as lactate and ammonia) accumulate. Harvest typically occurs at or soon after the point when the maximum cell density is achieved (typically 5-10×10⁶ cells/mL, depending on media formulation, cell line, etc.) typically around 3 to 7 days.

As used herein, the term “fed-batch culture” improves on the batch process by providing bolus or continuous media feeds to replenish those media components that have been consumed. Since fed-batch cultures receive additional nutrients throughout the culture process, they have the potential to achieve higher cell densities (>10 to 30×10⁶ cells/ml, depending on media formulation, cell line, etc.) and increased product titers, when compared to the batch method. Unlike the batch process, a biphasic culture can be created and sustained by manipulating feeding strategies and media formulations to distinguish the period of cell proliferation to achieve a desired cell density (the growth phase) from the period of suspended or slow cell growth (the production phase). As such, fed batch cultures have the potential to achieve higher product titers compared to batch cultures. As with the batch method, metabolic by-product accumulation will lead to declining cell viability over time as these progressively accumulate within the cell culture media, which limits the duration of the production phase to about 1.5 to 3 weeks. Fed-batch cultures are discontinuous and harvest typically occurs when metabolic by-product levels or the culture viability reach predetermined levels.

The term “polypeptide” or “protein” is used interchangeably herein with “amino acid residue sequences” and refers to a polymer of amino acids. These terms also include proteins that are post-translationally modified through reactions that include, but are not limited to, glycosylation, acetylation, phosphorylation or protein processing. Modifications and changes, for example fusions to other proteins, amino acid sequence substitutions, deletions or insertions, can be made in the structure of a polypeptide while the molecule maintains its biological functional activity. For example certain amino acid sequence substitutions can be made in a polypeptide or its underlying nucleic acid coding sequence and a protein can be obtained with the same properties. The terms also apply to amino acid polymers in which one or more amino acid residue is an analog or mimetic of a corresponding naturally occurring amino acid. The term “polypeptide” typically refers to a sequence with more than 10 amino acids and the term “peptide” to sequences with up to 10 amino acids in length.

The term “heterologous protein” as used herein refers to a polypeptide derived from a different organism or a different species from the host cell. The heterologous protein is coded for by a heterologous polynucleotide that is experimentally put into the host cell that does not naturally express that protein. A heterologous polynucleotide may also be referred to as transgene. Thus, it may be a gene or open reading frame (ORF) coding for a heterologous protein. The term “heterologous” when used with reference to a protein may also indicate that the protein comprises amino acid sequences that are not found in the same relationship to each other or the same length in nature. Thus, it also encompasses recombinant proteins. Heterologous may also refer to a polynucleotide sequence, such as a gene or transgene, or a portion thereof, being inserted into the mammalian cell's genome in a location in which it is not typically found. In the present invention the heterologous protein is preferably a therapeutic protein.

The term “medium”, “cell culture medium” and “culture medium” are used interchangeably herein and refer to a solution of nutrients that nourish cells, particularly mammalian cells. Cell culture media formulations are well known in the art. Typically a cell culture medium provides essential and non-essential amino acids, vitamins, energy sources, lipids and trace elements required by the cell for minimal growth and/or survival, as well as buffers, and salts. A culture medium may also contain supplementary components that enhance growth and/or survival above the minimal rate, including, but not limited to, hormones and/or other growth factors (such as insulin or insulin-like growth factor), particular ions (such as sodium, chloride, calcium, magnesium, and phosphate), buffers, vitamins, nucleosides or nucleotides, trace elements (inorganic compounds usually present at very low final concentrations) including trace metals, amino acids (including non-proteinogenic amino acids), lipids, anti-oxidants, glucose and/or other energy source, such as organic acids; as described herein. Also surfactants may be included in a medium. In certain embodiments, a medium is advantageously formulated to a pH and salt concentration optimal for cell survival and proliferation. A “cell culture perfusion medium” or “perfusion medium” is a medium used in continuous perfusion. The person skilled in the art will understand that further components not being part of the cell culture medium may be added to the cell culture during cultivation. For example anti-foaming agents may be added separately. Also glucose and/or glutamine may be added separately either exclusively or in addition to the glucose provided with the cell culture medium. Finally base (e.g., sodium carbonate or sodium hydroxide) may be added to the cell culture to control the pH during cultivation.

Examples for amino acids in cell culture media, without being limited thereto, are proteinogenic amino acids, such as glycine, alanine, arginine, asparagine, aspartic acid, cysteine, glutamic acid, glutamine, histidine, isoleucine, leucine, lysine, methionine, phenylalanine, proline, serine, threonine, tryptophan, tyrosine, and valine and salts or derivatives thereof as well as non-proteinogenic amino acids such as hydroxyproline, ornithine, α-amino-n-butyric acid and salts an derivatives thereof. Derivatives thereof include for example cystine, the oxidized dimer of cysteine, or dipeptides, preferably alanyl- or glycyl-dipeptides of amino acids, such as glutamine, tyrosine or cysteine. Examples for inorganic salts, without being limited thereto are calcium chloride, magnesium chloride, magnesium sulfate, potassium chloride, sodium bicarbonate, sodium chloride, sodium phosphate, sodium meta-silicate, trace metal salts etc. and hydrates thereof. Examples for trace metals, without being limited thereto, are zinc, copper, chromium, nickel, cobalt, vanadium, molybdene and manganese and salts thereof, such as ammonium molybdate, cupric sulfate, sodium selenite, manganese chloride, manganese sulfate, zinc chloride, zinc sulfate etc. and hydrates thereof. Examples for iron sources, without being limited thereto, are ferric citrate, ferric nitrate, ferrous sulfate, ferrous chloride, ferric chloride, ferrous phosphate. Examples of vitamins, without being limited thereto, are biotin, choline chloride, choline, pantothenate, D-calcium, folic acid, niacinamide, p-aminobenzoic acid, pyridoxal, pyridoxine, riboflavin, thiamine, tocopherol, vitamin B12, retinols (Vitamin A), ascorbate etc. and salts thereof. Examples of polyamines are, without being limited thereto, putrescine, spermidine and spermine, organic acids may be taurine or alternative carbon sources, such as succinic acid, pyruvate, citric acid, fatty acids may be linoleic acid, linolenic acid, palmitic acid, and oleic acid, a surfactant may be pluronic F68, buffers may be for example phosphate buffers (monobasic phosphate salts and dibasic phosphate salts), anti-oxidants may be for example reduced glutathione or lipoic acid, and examples for chelators are without being limited thereto citrate or ethylenediaminetetraacetic acid (EDTA). Energy sources may be pyruvate or dextrose etc. Other compounds that may be present in a medium are ethanolamine, taurine, i-inositol and proteins such as insulin or insulin-like growth factor. Compounds may also be added for formulating the dry powder medium, such as dextrose may be added for milling purposes only and not as medium component.

The medium according to the invention is a serum-free perfusion culture medium (or serum-free cell culture perfusion medium) that is added 0 to 4 days post-inoculation, i.e., the perfusion culture starts from day 0 to day 4 of the cell culture. It may therefore also be referred to as cell culture perfusion medium feed, as it is typically added following inoculation. Perfusion cell culture, may have different phases of culturing, including a growth phase and a production phase. The particular medium used during growth phase (growth medium) and production phase (production medium) may be particularly designed for implementation in said specific phase. Typically cells are inoculated in a growth medium before perfusion with a production medium begins. Also perfusion may already begin before replacing the growth medium with a production medium. In certain embodiments, the cell culture medium according to the invention is a production medium. However, both media, the growth medium and the production medium, are complete media and allow maintenance and/or growth of the cell culture (i.e., without the need for being mixed with a further medium). This is in contrast to a feed medium or fed-batch medium used in fed-batch culture, which is typically an incomplete medium replenishing consumed nutrients, but components such as salts and buffers are typically reduced to reduce the osmolality of the medium and to allow further concentration of the feed medium. The medium would typically not be sufficient to support cell culture maintenance without being mixed with the basal medium or inoculation medium.

The term “perfusion medium” refers to a solution of nutrients that nourish cells, particularly mammalian cells and is used in perfusion culture. It may be a growth medium and/or a production medium. It is typically designed to support perfusion cultures during production phase. As it provides a steady source of fresh nutrients and is constantly removed from the bioreactor, the perfusion medium is a complete medium that allows maintenance and/or growth of the cell culture. The term “complete medium” refers to a solution of nutrients that contains all components of the medium intended to be present in the cell culture.

The serum-free cell culture perfusion medium according to the invention is a complete medium and may be present in a compartmentalized form comprising at least three separate aqueous concentrated feeds and a diluent, wherein the first concentrated feed is an alkaline concentrated feed, the second concentrated feed is an acidic concentrated feed and the third concentrated feed is a neutral concentrated feed, or upon mixing as the resulting serum-free cell culture perfusion medium. The term “serum-free cell culture perfusion medium” without the explicit characterization that the medium is compartmentalized refers to the resulting serum-free cell culture perfusion medium formed upon mixing. Since the compartmentalized medium is for direct addition to the cell culture and/or the reaction vessel of the bioreactor, the resulting serum-free cell culture medium typically does not exist in a pure or isolated from, but is rather a mixture with the already present cell culture, i.e., culture medium and cells. It is therefore important that the compartmentalized medium is pH-adjusting upon mixing. However, since the pH in the culture may vary during cultivation of cells pH adjustment using base during culture may still be necessary to maintain a constant pH.

The term “serum-free” as used herein refers to a cell culture medium that does not contain animal or human serum, such as fetal bovine serum. Preferably serum-free medium is free of proteins isolated from any animal or human derived serum. Various tissue culture media, including defined culture media, are commercially available, for example, any one or a combination of the following cell culture media can be used: RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series (JRH Biosciences, Lenexa, Kans.), among others. Serum-free versions of such culture media are also available. Cell culture media may be supplemented with additional or increased concentrations of components such as amino acids, salts, sugars, vitamins, hormones, growth factors, buffers, antibiotics, lipids, trace elements and the like, depending on the requirements of the cells to be cultured and/or the desired cell culture parameters.

The term “protein-free” as used herein refers to a cell culture medium that does not contain any protein. Thus, it is devoid of proteins isolated from an animal or human, derived from serum or recombinantly produced proteins, such as recombinant proteins produced in mammalian, bacterial, insect or yeast cells. A protein-free medium may contain single recombinant proteins, such as insulin or insulin-like growth factor, but only if this addition is explicitly stated.

As used herein the term “chemically defined” refers to a culture medium, which is serum-free and which does not contain any hydrolysates, such as protein hydrolysates derived from yeast, plants or animals. Preferably a chemically defined medium is also protein-free or contains only selected recombinantly produced (not animal derived) proteins, such as recombinant insulin and/or recombinant insulin-like growth factor. Chemically defined medium consist of a mixture of characterized and purified substances. An example of a chemically defined medium is for example CD-CHO medium from Invitrogen (Carlsbad, Calif., US).

The term “suspension cells” or “non-adherent cells” as used herein relates to cells that are cultured in suspension in liquid medium. Adhesive cells such as CHO cells may be adapted to be grown in suspension and thereby lose their ability to attach to the surface of the vessel or tissue culture dish.

As used herein, the term “bioreactor” means any vessel useful for the growth of a cell culture. A bioreactor can be of any size as long as it is useful for the culturing of cells; typically, a bioreactor is sized appropriate to the volume of cell culture being grown inside of it. Typically, a bioreactor will be at least 1 liter and may be 2 or more, 5 or more, 10 or more, 50 or more, 100 or more, 200 or more, 250 or more, 500 or more, 1,000 or more, 1,500 or more, 2,000 or more, 2,500 or more, 5,000 or more, 8,000 or more, 10,000 or more, 12,000 or more liters. Preferably the bioreactor will be at least 100 liters, more preferably at least 1,000 liters. The internal conditions of the bioreactor, including, but not limited to pH and temperature, can be controlled during the culturing period. Those of ordinary skill in the art will be aware of, and will be able to select, suitable bioreactors for use in practicing the present invention based on the relevant considerations. The cell cultures used in the methods of the present invention can be grown in any bioreactor suitable for perfusion culture. The particular type of bioreactor is not particularly limited and may encompass all types of bioreactors suitable for perfusion cell culture.

As used herein, “cell density” refers to the number of cells in a given volume of culture medium. “Viable cell density” refers to the number of live cells in a given volume of culture medium, as determined by standard viability assays (such as trypan blue dye exclusion method).

As used herein, the term “cell viability” means the ability of cells in culture to survive under a given set of culture conditions or experimental variations. The term as used herein also refers to that portion of cells which are alive at a particular time in relation to the total number of cells, living and dead, in the culture at that time.

As used herein, the term “titer” means the total amount of a polypeptide or protein of interest (which may be a naturally occurring or recombinant protein of interest) produced by a cell culture in a given amount of medium volume. Titer can be expressed in units of milligrams or micrograms of polypeptide or protein per milliliter (or other measure of volume) of medium.

As used herein, the term “yield” refers to the amount of heterologous protein produced in perfusion culture over a certain period of time. The “total yield” refers to the amount of heterologous protein produced in perfusion culture over the entire run.

The term “reduction”, “reduced”, “reduce” or “lower” as used herein, generally means a decrease by at least 5% as compared to a reference level, for example a decrease by at least 10% as compared to a reference level, or at least about 20%, or at least about 30%, or at least about 40%, or at least about 50%, or at least about 60%, or at least about 70%, or at least about 75%, or at least about 80%, or at least about 90% or up to and including a 100% decrease, or any integer decrease between 10-100% as compared to a control mammalian cell culture, which is cultured under the same conditions using the same serum-free cell culture medium, such as wherein the osmolality is not increased during culture, particularly during perfusion culture.

The term “enhancement”, “enhanced”, “enhanced”, “increase”, or “increased”, as used herein, generally means an increase by at least 5% as compared to a control cell, for example an increase by at least about 10%, or at least about 20%, or at least about 30%, or at least about 40%, or at least about 50%, or at least about 75%, or at least about 80%, or at least about 90%, or at least about 100%, or at least about 200%, or at least about 300%, or any integer decrease between 10-300% as compared to a control mammalian cell culture, which is cultured under the same conditions using the same serum-free cell culture medium, such as wherein the osmolality is not increased during culture, particularly during perfusion culture.

As used herein, a “control cell culture” or “control mammalian cell culture” is a cell culture which is the same as the cell culture to which it is compared to, using the same serum-free cell culture medium comprising the medium components subgroup into at least three aqueous concentrated feeds and a diluent according to the invention except that the osmolality is not increased during culture, particularly during perfusion culture.

The term “mammalian cells” as used herein are cells lines suitable for the production of a heterologous protein, preferably a therapeutic protein, more preferably a secreted recombinant therapeutic protein. Preferred mammalian cells according to the invention are rodent cells such as hamster cells. The mammalian cells are isolated cells or cell lines. The mammalian cells are preferably transformed and/or immortalized cell lines. They are adapted to serial passages in cell culture and do not include primary non-transformed cells or cells that are part of an organ structure. Preferred mammalian cells are BHK21, BHK TK; CHO, CHO-K1, CHO-S cells, CHO-DXB11 (also referred to as CHO-DUKX or DuxB11), and CHO-DG44 cells or the derivatives/progenies of any of such cell line. Particularly preferred are CHO-DG44, CHO-K1 and BHK21, and even more preferred are CHO-DG44 and CHO-K1 cells. Most preferred are CHO-DG44 cells. Glutamine synthetase (GS)-deficient derivatives of the mammalian cell, particularly of the CHO-DG44 and CHO-K1 cell are also encompassed. The mammalian cell may further comprise one or more expression cassette(s) encoding a heterologous protein, preferably a recombinant secreted therapeutic protein. The mammalian cells may also be murine cells such as murine myeloma cells, such as NS0 and Sp2/0 cells or the derivatives/progenies of any of such cell line. However, derivatives/progenies of those cells, other mammalian cells, including but not limited to human, mice, rat, monkey, and rodent cell lines, can also be used in the present invention, particularly for the production of biopharmaceutical proteins.

The term “growth phase” as used herein refers to the phase of cell culture where the cells proliferate exponentially and viable cell density in the bioreactor is increasing. Cells in culture usually proliferate following a standard growth pattern. After the culture is seeded there may be a lag phase, which is a period of slow growth when the cells are adapting to the culture environment and preparing for fast growth. The growth phase (also referred to as log phase or logarithmic phase) is a period where the cells proliferate exponentially and consume the nutrients of the growth medium. It is followed by the production phase.

The term “production phase” refers to the phase of cell culture which begins once harvest is started, which may be at or before the target viable cell density is reached. Harvest is typically started when the heterologous protein reaches about 0.2 gram/L_{bioreactor}/day in the permeate. A typical target cell density is in the range of about 10×10⁶ cells/ml to about 120×10⁶ cells/ml, but may be even higher. Thus, the target cell density according to the present invention is at least at least 30×10⁶ cells/ml, at least 40×10⁶ cells/ml, at least 50×10⁶ cells/ml, at least 60×10⁶ cells/ml, at least 80×10⁶ cells/ml or at least 100×10⁶ cells/ml. The target cell density may even be as high as 100×10⁶ cells/ml to 200×10⁶ cells/ml, preferably about 120×10⁶ cells/ml to 150×10⁶ cells/ml.

In certain embodiments herein, the osmolality of the cell culture is increased to a level resulting in growth suppression at the start of production phase. Preferably osmolality increased gradually or stepwise from a level optimal for growth. Thus, osmolality needs to be increased before the target viable cell density is reached. Preferably osmolality is increased gradually or stepwise from a level optimal for growth to a level resulting in growth suppression starting at about half the target viable cell density. This allows that an osmolality level resulting in growth suppression is reached once the target viable cell density is reached. It is important that high osmolality (i.e., an osmolality level resulting in growth suppression) is maintained until the end of the culture. The person skilled in the art will understand that removing osmotic pressure will remove growth inhibition.

The term “growth-arrest”, “growth inhibition” and “growth suppression” are used synonymously herein and refer to cells that are stopped from increasing in number, i.e., from cell division. The cell cycle comprises the interphase and the mitotic phase. The interphase consists of three phases: DNA replication is confined to S phase; G₁ is the gap between M phase and S phase, while G₂ is the gap between S phase and M phase. In M phase, the nucleus and then the cytoplasm divide. In the absence of a mitogenic signal to proliferate or in the presence of compounds that induce growth arrest the cell cycle arrests. The cells may partly disassemble their cell-cycle control system and exit from the cycle to a specialized, non-dividing state called G₀. Growth suppression can be easily assessed by determining the viable cell density over time. Preferably cells are maintained at a viable cell density with a variation of 30%, more preferably 20%. More preferably cells are maintained at the target viable cell is maintained at density with a variation of 30%, more preferably 20%.

### Cell Culture Perfusion Medium

In one aspect of the disclosure, a compartmentalized serum-free cell culture perfusion medium comprising the medium components subgrouped into at least three separate aqueous concentrated feeds and a diluent is provided, wherein the first concentrated feed is an alkaline concentrated feed, the second concentrated feed is an acidic concentrated feed and the third concentrated feed is a near neutral concentrated feed; wherein the compartmentalized serum-free cell culture perfusion medium is pH-adjusting to neutral pH upon mixing of the at least three separate aqueous concentrated feeds and the diluent in the resulting serum-free cell culture perfusion medium. In a preferred embodiment the at least three separate aqueous concentrated feeds are not premixed prior to addition to the cell culture and/or the reaction vessel of the bioreactor. Premixing of two or more feeds is not ideal, as precipitation upon mixing may occur. Thus, the compartmentalized serum-free cell culture perfusion medium is suitable for separate addition of the alkaline concentrated feed, the acidic concentrated feed and the near neutral concentrated feed to a cell culture and/or a reaction vessel of a bioreactor; direct addition of the alkaline concentrated feed, the acidic concentrated feed and the near neutral concentrated feed to a cell culture and/or a reaction vessel of a bioreactor without prior pre-mixing; and/or direct mixing of the at least three separate aqueous concentrated feeds in a cell culture and/or a reaction vessel of a bioreactor. In a preferred embodiment the serum-free cell culture perfusion medium comprises the medium components subgrouped into at least three separate aqueous concentrated feeds as described and a diluent. This includes a serum-free cell culture perfusion medium consisting of the at least three separate aqueous concentrated feeds and a diluent. The medium components are primarily distributed according to their intrinsic properties, such as solubility at neutral pH and/or improved solubility at alkaline or acidic pH. In a preferred embodiment the serum-free cell culture perfusion medium is a production medium. The person skilled in the art will understand that perfusion culture is typically performed using mammalian cells, thus the perfusion culture medium is a perfusion culture medium for mammalian cells.

The person skilled in the art will also understand that further components not being part of the cell culture medium may be added to the cell culture during cultivation. For example anti-foaming agents may be added separately. Also glucose and/or glutamine feeds may be added separately either exclusively or in addition to the glucose and/or glutamine provided with the cell culture medium. Finally base (e.g., sodium carbonate or sodium hydroxide) may be added to the cell culture to control the pH during cultivation.

In one embodiment, the serum-free cell culture perfusion medium may be chemically defined and/or hydrolysate-free. Hydrolysate-free means that the medium does not contain protein hydrolysates from animal, plant (soybean, potato, rice), yeast or other sources. Typically a chemically defined medium is hydrolysate-free. In any case the serum-free perfusion medium should be free of compounds derived from animal sources, particularly proteins or peptides derived and isolated from an animal (this does not include recombinant proteins produced by the cell culture). Preferably the serum-free cell culture perfusion medium is protein-free or protein-free except for recombinant insulin and/or insulin-like growth factor. Thus, the serum-free cell culture medium may be a protein-free medium or a protein-free medium comprising recombinant insulin and/or recombinant insulin-like growth factor. The person skilled in the art will understand that a protein-free medium is typically chemically defined and/or hydrolysate-free. More preferably the serum-free cell culture perfusion medium is chemically defined and protein-free or protein-free except for recombinant insulin and/or insulin-like growth factor. This also applies to the serum-free culture perfusion medium used in the methods or prepared according to the methods of the present invention. In case an initial growth medium and a production medium is used this applies to both media.

To adjust the compartmentalized serum-free cell culture perfusion medium to the desired “working” concentration an appropriate volume of each of the at least three separate aqueous concentrated feeds at a ratio determined by their fold-concentrations relative to each other and diluted with an appropriate amount of the diluent are mixed to provide the serum-free cell culture perfusion medium, i.e., the serum-free cell culture perfusion medium at working concentration. Although the diluent used in the serum-free cell culture perfusion medium according to the invention may theoretically also be an aqueous saline solution and/or an aqueous buffer, it is preferably sterile water. Sterile water is advantageous as it does not need to be prepared or mixed and hence avoids the need for additional storage space for premade components. The ratio (v/v) of the diluent to the cumulative volume of the at least three separate aqueous concentrated feeds added to the cell culture and/or the reaction vessel of the bioreactor to provide the resulting serum-free cell culture perfusion medium that is pH-adjusting to a near neutral pH determines the fold-concentration of the serum-free cell culture perfusion medium in the cell culture and/or the reaction vessel of the bioreactor. Thus, the advantage of using the at least three separate aqueous concentrated feed is that the fold-concentration of the medium may be adapted to the viable cell density and the nutritive needs (maintain nutritive balance). Osmolality may be used as a surrogate to estimate nutritive balance in and out of the system. Thus, an osmo balance may be used to calculate adjustment of the cumulative volume of the concentrated feeds (at their fixed ratio to each other) and diluent feed rates to achieve a desirable residual osmolality and nutritive level.

The resulting serum-free cell culture perfusion medium is pH-adjusting to neutral pH upon mixing of the at least three separate aqueous concentrated feeds and the diluent. This means the pH is automatically adjusted upon mixing without the need for addition of a titrant such as NaOH or HCl. The pH of the culture medium should be neutral at a pH of between about 6.7 and about 7.5, preferably between about 6.9 and about 7.4, and more preferably between about 6.9 and about 7.2, upon mixing of the at least three separate aqueous concentrated feeds and the diluent.

The alkaline concentrated feed may be a 2× to 80× concentrated feed, preferably a 20× to 40× concentrated feed, more preferably a 20× to 30× concentrated feed and most preferably a 25× feed. Generally a higher concentrated feed is preferred. However, for optimal results for example the alkaline concentrated feed may be prepared as a concentrated feed that is not maximally concentrated to better match the near neutral and/or acidic feed. This also safes titrant in the concentrated feed, such as the alkaline concentrated feed. The near neutral concentrated feed may be a 2× to 50× concentrated feed, preferably a 10× to 40× concentrated feed, more preferably a 20× to 30× concentrated feed and most preferably a 25× concentrated feed. The acidic concentrated feed may be a 2× to 40× concentrated feed, a 4× to 20× concentrated feed, a 5× to 12× concentrated feed or a 6× to 10× concentrated feed. Generally a higher concentrated feed (alkaline, acidic and neutral, combined and individually) is preferred. However, for optimal results for example the alkaline concentrated feed may be prepared as a concentrated feed that is not maximally concentrated (e.g., less than 80×) to better match the near neutral and/or acidic feed. This also safes titrant in the concentrated feed, such as the alkaline concentrated feed.

In one embodiment the alkaline concentrated feed is a 2× to 80× concentrated feed, the acidic concentrated feed is a 2× to 40× concentrated feed and the near neutral concentrated feed is a 2× to 50× concentrated feed, preferably the alkaline concentrated feed is a 20× to 40× concentrated feed, the acidic concentrated feed is a 4× to 20× concentrated feed and the near neutral concentrated feed is a 10× to 40× concentrated feed, more preferably alkaline concentrated feed is a 20× to 30× concentrated feed, the acidic concentrated feed is a 5× to 12× concentrated feed and the near neutral concentrated feed is a 20× to 30× concentrated feed and most preferably the alkaline concentrated feed is a 25× concentrated feed, the acidic concentrated feed is a 6× to 10× concentrated feed and the near neutral concentrated feed is a 25× concentrated feed. In a specific embodiment the alkaline concentrated feed and the near neutral concentrated feed are about similarly concentrated. Thus for example the alkaline concentrated feed is a 20× to 30× concentrated feed and the near neutral concentrated feed is a 20× to 30× concentrated feed or the alkaline concentrated feed is a 25× concentrated feed and the near neutral concentrated feed is a 25× concentrated feed and the acidic concentrated feed is maximally concentrated.

In the serum-free cell culture perfusion medium, the ratio (v/v/v) of the alkaline concentrated feed to the acidic concentrated feed to the near neutral concentrated feed is a fixed ratio to provide the resulting serum-free cell culture perfusion medium that is pH-adjusting to a neutral pH. Thus, the at least three concentrated feeds are added at a fixed ratio (v/v/v) to each other depending on their fold-concentration to maintain the relative proportion of the medium components in the 1× serum-free cell culture perfusion medium (1× formulation). In other words, the ratio of feeds to each other should be such that the original ratios from the 1× formulation are maintained. For example in case the alkaline concentrated feed is a 25× concentrated feed, the acidic concentrated feed is a 6× concentrated feed and the near neutral concentrated feed is a 25× concentrated feed and concentrated feeds are added at a ratio of 1:4.2:1 or in case the alkaline concentrated feed is a 30× concentrated feed, the acidic concentrated feed is a 10× concentrated feed and the near neutral concentrated feed is a 30× concentrated feed and concentrated feeds are added at a ratio of 1:3:1. Further, the ratio (v/v) of the diluent to the cumulative volume of the at least three separate aqueous concentrated feeds in the resulting serum-free cell culture perfusion medium that is pH-adjusting to a neutral pH determines the osmolality of the serum-free cell culture perfusion medium. The ratio (v/v) of the diluent to the cumulative volume of the at least three separate aqueous concentrated feeds in the serum-free cell culture perfusion medium that is pH-adjusting to a neutral pH also determines the fold-concentration of the serum-free cell culture perfusion medium. The fold-concentration could be anything from 0.1× to the maximal fold-concentration, but is typically between 0.5× and 2×, preferably between 1× and 2×. The maximal fold-concentration (nₘₐₓ X) upon mixing of the three separate aqueous concentrated feeds may be calculated as follows:

nₘₐₓX=(nₐₗₖₐₗᵢₙₑX*n_{acidic} X*nₙₑᵤₜᵣₐₗ((nₐₗₖₐₗᵢₙₑ X*n_{acidic}(nₐₗₖₐₗᵢₙₑ X*nₙₑᵤₜᵣₐₗ X)+(n_{acidic} X*nₙₑᵤₜᵣₐₗ X)),

wherein
nₘₐₓ X is the maximal fold-concentration upon mixing of the three separate aqueous concentrated feeds;
nₐₗₖₐₗᵢₙₑ X is the n-fold concentration of the alkaline concentrated feed;
n_{acidic} X is the n-fold concentration of the acidic concentrated feed;
nₙₑᵤₜᵣₐₗ X is the n-fold concentration of the near neutral concentrated feed; and
* denotes the mathematical operation multiplication.
For example in case the alkaline concentrated feed is a 25× concentrated feed, the acidic concentrated feed is a 6× concentrated feed and the near neutral concentrated feed is a 25× concentrated feed the maximal fold-concentration upon mixing of the three separate aqueous concentrated feeds is 4.1×. Thus the reduction in prepared medium consumption is about 75%. In case the alkaline concentrated feed is a 30× concentrated feed, the acidic concentrated feed is a 10× concentrated feed and the near neutral concentrated feed is a 30× concentrated feed the maximal fold-concentration upon mixing of the three separate aqueous concentrated feeds is 6×. Thus the reduction in prepared medium consumption is more than 80%. Also using concentrated feeds allow to adjust the fold-concentration of the serum-free cell culture medium in the cell culture and/or bioreactor and hence allows maintenance of higher viable cell densities at a similar or only moderately increased perfusion rate and consequently at a reduced cell specific perfusion rate.

The serum-free cell culture perfusion medium comprises an alkaline concentrated feed, an acidic concentrated feed and a near neutral concentrated feed. Near neutral concentrated feed refers to a pH of 7.5±1.0. Thus the near neutral concentrated feed has a pH of about 6.5 to about 8.5. The near neutral concentrated feed preferably does not contain any additional titrants. Avoidance of titrants saves osmo space in the resulting serum-free cell culture perfusion medium. Thus, the pH of the near neutral concentrated feed may be slightly alkaline at a pH up to about 8.5. Preferably the near neutral pH has a pH of about 7 to about 8.5, more preferably of about 7.5 to about 8.5.

The alkaline concentrated feed may have a pH of about 9 or higher, such as a pH of about 9 to about 11, preferably a pH of about 9.8 to about 10.8, more preferably a pH of about 9.8 to about 10.5. The acidic concentrated feed may have a pH about of 5 or lower, such as a pH of pH of about 2 to about 5, preferably a pH of about 3.6 to about 4.8 and more preferably a pH of about 3.8 to about 4.5. Although a pH may be adjusted rather precisely a typical pH variation is a variation of 0.5.

In one embodiment the alkaline concentrated feed has a pH of about 9 or higher, the acidic concentrated feed has a pH of about 5 or lower and the near neutral concentrated feed has a pH of about 7 to about 8.5. Preferably, the alkaline concentrated feed has a pH of about 9 to about 11, the acidic concentrated feed has a pH of about 2 to about 5 and the near neutral concentrated feed has a pH of about 7 to about 8.5; more preferably the alkaline concentrated feed has a pH of about 9.8 to about 10.8, the acidic concentrated feed has a pH of about 3.6 to about 4.8 and the near neutral concentrated feed has a pH of about 7 to about 8.5; and most preferably the alkaline concentrated feed has a pH of about 9.8 to about 10.5, the acidic concentrated feed has a pH of about 3.8 to about 4.5 and the near neutral concentrated feed has a pH of about 7.5 to about 8.5.

The medium components are primarily distributed according to their intrinsic properties, such as solubility at neutral pH and/or improved solubility at alkaline or acidic pH. Furthermore medium components that are particularly insoluble in aqueous solution may be split into separate feeds. For example choline chloride may be provided with the near neutral concentrated feed and the acidic concentrated feed in order to achieve the required concentrations in the final serum-free cell medium.

The near neutral concentrated feed preferably comprises all vitamins soluble at neutral pH. Further, the near neutral concentrated feed preferably does not contain any metals. Since metals may interact with some vitamins, vitamins are preferably kept separate from metals. Vitamins are therefore provided preferably in the near neutral concentrated feed and alternatively in the acidic concentrated feed. One exception is folic acid, which may also be provided with the alkaline feed. Thus, in one embodiment vitamins and metals are provided in separate feeds, preferably vitamins are in the near neutral feed and metals are in the acidic feed. However, vitamins poorly soluble in aqueous solutions at neutral pH may also be in the acidic feed. For examples vitamins such as pantothenate, thiamine, choline chloride and/or pyridoxine may also be provided in the acidic feed. Furthermore vitamins that are generally poorly soluble in aqueous solutions, such as choline chloride, may be present in the neutral feed and the acidic feed. The neutral concentrated feed may also comprise compounds such as L-α-amino-n-butyric acid, i-inositol and/or the fatty acid linoleic acid. Furthermore, bicarbonate is preferably provided with the neutral concentrated feed. In one embodiment the neutral concentrated feed does not contain any additional titrant for pH adjustment.

Salts and metals are preferably provided in the acidic concentrated feed. For example and without being limited thereto the acidic concentrated feed may comprise trace elements, trace metals, inorganic salts, iron sources chelators, polyamines, and/or regulatory hormones, such as insulin or insulin-like growth factor. Amino acids selected from the group consisting of alanine, arginine, asparagine, glutamic acid, glutamine, glycine, isoleucine, leucine, lysine, methionine, phenylalanine, proline, serine, threonine, tryptophan and valine are preferably in the acidic concentrated feed. Furthermore, surfactants, anti-oxidants, and carbon sources, and optionally also ethanolamine and/or fatty acids may be provided in the acidic concentrated feed and/or the near neutral concentrated feed.

The alkaline concentrated feed primarily comprises amino acids with maximum solubility at alkaline pH of about 9 or higher. Preferably the alkaline concentrated feed comprises at least aspartic acid, histidine, tyrosine and cysteine. Cysteine is water soluble, but readily oxidizes to cystine with poor water solubility at neutral pH. Thus, cysteine and/or cystine are preferably in the alkaline concentrated feed. Another compound soluble at an alkaline pH of about 9 or higher is folic acid. Thus, folic acid may also be provided with the alkaline feed. Amino acids that are not provided with the alkaline concentrated feed are preferably provided with the acidic concentrated feed. Thus, in one embodiment the remaining amino acids (i.e., amino acids that do not have a maximum solubility at alkaline pH of about 9 or higher and/or amino acids that are not provided with the alkaline feed) are provided in the acidic and/or near neutral concentrated feed, preferably in the acidic concentrated feed.

The person skilled in the art will understand that a complete medium is more difficult to provide as a concentrate compared to, e.g., a feed-medium for a fed-batch culture as it comprises more components, particularly salts and buffers that increase osmolality and hence restrict the osmo space. Also modern nutritive rich media are more difficult to provide as concentrate compared to prior art media, such as RPMI 1640 and DMEM/F12 and others. These more modern nutritive rich media are particularly rich in amino acids typically comprising amino acids in a mM range rather than in a μM range. The serum-free cell culture perfusion medium according to the invention is therefore a medium comprising amino acids at more than 50 mM, preferably more than 70 mM, more preferably more than 100 mM, even more preferably more than 120 mM in a 1× serum-free cell culture perfusion medium. Since glutamine is sometimes added separately the serum-free cell culture perfusion medium preferably comprises natural amino acids except for glutamine at more than 50 mM, preferably more than 70 mM, more preferably more than 100 mM, even more preferably more than 120 mM in the resulting serum-free cell culture perfusion medium. Natural amino acids except for glutamine refer to alanine, glycine, arginine, asparagine, aspartic acid, cysteine, glutamic acid, histidine, isoleucine, leucine, lysine, methionine, phenylalanine, proline, histidine, serine, threonine, tryptophan, tyrosine and valine. However, not all natural amino acids need to be present in the serum-free cell culture perfusion medium, such as e.g., alanine and glycine. Natural amino acids also include derivatives of a natural amino acid such as dipeptides or cystine.

The person skilled in the art is used to optimize individual processes with regard to cell culture media compositions as well as for other process characteristics and culture performance. For example and especially where very high cell densities are not material they can be tested in shake flasks. In cases where higher oxygenation rates are intended spin tubes (as disclosed e.g. in Strnad et al., Biotechnol. Prog., 2010, Vol. 26, No. 3, pages 653-663) can be used, which agitate at higher rotations per minute (rpm). Spin tube bioreactors can advantageously be used as a small scale model for evaluation of media, various process parameters, and growth characteristics at high density (>20e6 c/mL). They can also reduce the time and effort required for process development by alleviating the need for large media preps and operation of bench-scale bioreactors. The ability to centrifuge multiple Spin tubes to perform media exchanges enables perfusion cell culture at small scale (working volume 15 mL).

The at least three separate aqueous concentrated feeds are preferably sterile prior to storage and prior to mixing. In one embodiment the at least three separate aqueous concentrated feeds are filter sterilized. In addition, regarding the mixing of the components the at least three separate aqueous concentrated feeds are not premixed prior to addition of the cell culture and/or the reaction vessel of the bioreactor. Thus, preferably the aqueous concentrated feeds are added directly to the cell culture and/or the reaction vessel of the bioreactor, preferably through separate entry points. The entry point may be a valve or a port in the bioreactor. Preferably the at least three separate aqueous concentrated feeds are added dropwise. It is advantageous that the at least three separate aqueous concentrated feeds are added continuously at a predetermined perfusion rate and hence simultaneously. They may be added from the bottom, from the top or from the side of the bioreactor and adjacent to each other or at different sides as long as the culture is continuously mixed.

The diluent (e.g., sterile water) may be added separately to the cell culture and/or the reaction vessel of the bioreactor. Thus, preferably the diluent is added directly to the cell culture and/or the reaction vessel of the bioreactor, preferably through an entry point separate to the entry points of the at least three separate aqueous concentrated feeds. The entry point may be a valve or a port in the bioreactor. It is advantageous that the diluent is added continuously at a predetermined perfusion rate and hence simultaneously with the at least three separate aqueous concentrated feeds. It may be added from the bottom, from the top or from the side of the bioreactor and adjacent to one or all of the at least three separate aqueous concentrated feeds or at a different side as long as the culture is continuously mixed. Alternatively the diluent may be premixed with one of the at least three separate aqueous concentrated feeds immediately before addition to the cell culture and/or the reaction vessel of the bioreactor. Thus, the diluent may be added to the cell culture and/or the reaction vessel of the bioreactor with one of the at least three separate aqueous concentrated feeds, preferably through entry points separate to the at least two other separate aqueous concentrated feeds. In one embodiment the diluent is premixed with the alkaline concentrated feed immediately before addition to the cell culture and/or the reaction vessel of the bioreactor.

In another aspect the present invention also relates to the use of the compartmentalized serum-free cell culture perfusion medium according to the invention for culturing mammalian cells, preferably in a perfusion culture. In one embodiment the cell culture medium according to the invention is used for controlling osmolality in a cell culture, preferably in a perfusion cell culture. Particularly the osmolality is increased in a perfusion cell culture. Increasing the osmolality in the cell culture suppresses cell growth and increases heterologous protein production. By increasing the osmolality in the cell culture, cell growth may be suppressed to maintain a sustainable viable cell density without cell bleeding, which may also be referred to as a dynamic perfusion culture.

By increasing the osmolality in the cell culture, the yield of the heterologous protein produced in the cell culture may be increased by at least about 5%, at least about 10% at least about 25%, at least about 50%, at least about 75%, at least about 100 percent, or about 5-50%, preferably about 10 to 100% relative to the yield in a control cell culture, wherein the osmolality is not increased. Preferable the yield is determined for a part or the entire culture period.

By using the serum-free cell culture medium according to the invention or the serum-free cell culture medium obtained by the method according to the invention and optionally further increasing the osmolality in the cell culture, the cell specific perfusion rate (pl/cell/day) reduced by at least about 25%, at least 30%, or at least about 50%, relative to the cell specific perfusion rate of a 1× serum-free cell culture medium. The cell specific perfusion rate (pl/cell/day) of the serum-free cell culture perfusion medium according to the invention or the serum-free cell culture medium obtained by the method according to the invention is preferably constant for a part or the entire culture period.

The invention also relates to an alkaline aqueous concentrated feed for combination with an acidic aqueous concentrated feed, a near neutral aqueous concentrated feed and a diluent to form a serum-free cell culture perfusion medium, wherein the pH of the serum-free cell culture perfusion medium is automatically adjusted to a neutral pH. In another embodiment the invention relates to an acidic aqueous concentrated feed for combination with an alkaline aqueous concentrated feed, a near neutral aqueous concentrated feed and a diluent to form a serum-free cell culture perfusion medium, wherein the pH of the resulting serum-free cell culture perfusion medium is automatically adjusted to a neutral pH. In yet another aspect the invention relates to a near neutral aqueous concentrated feed for combination with an alkaline aqueous concentrated feed, an acidic aqueous concentrated feed and a diluent to form a serum-free cell culture perfusion medium, wherein the pH of the resulting serum-free cell culture perfusion medium is automatically adjusted to a neutral pH. Wherein the alkaline aqueous concentrated feed, the acidic aqueous concentrated feed, the near neutral aqueous concentrated feed, the diluent and the serum-free cell culture perfusion medium may be further characterized as disclosed above.

### Method of Preparing a Serum-Free Cell Culture Perfusion Medium

In yet another aspect, the invention relates to a method of preparing a serum-free cell culture perfusion medium comprising comprising (a) providing the components of a cell culture media in at least three subgroups of components based on solubility at alkaline, acidic and neutral pH, (b) providing (i) the subgroup of components soluble at alkaline pH in an alkaline aqueous solution to form an alkaline concentrated feed; (ii) the subgroup of components soluble at acidic pH in an acidic aqueous solution to form an acidic concentrated feed; and (iii) the subgroup of components soluble at neutral pH in a neutral aqueous solution to form a near neutral concentrated feed; (c) optionally storing the prepared alkaline concentrated feed, acidic concentrated feed and near neutral concentrated feed in separate containers; and (d) adding the prepared alkaline concentrated feed, acidic concentrated feed and near neutral concentrated feed and the diluent to the cell culture and/or the reaction vessel of the bioreactor, wherein (i) the alkaline concentrated feed, the acidic concentrated feed and the near neutral concentrated feed are added separately to the cell culture and/or the reaction vessel of the bioreactor; and (ii) the diluent is added separately to the cell culture and/or the reaction vessel of the bioreactor or the diluent is premixed with one of the at least three separate aqueous concentrated feeds immediately before addition to the cell culture and/or the reaction vessel of the bioreactor; wherein the pH of the resulting serum-free cell culture perfusion medium is automatically pH adjusted to a near neutral pH upon mixing of the at least three separate aqueous concentrated feeds and the diluent. Thus, the serum-free cell culture perfusion medium prepared according to the method comprises the medium components subgrouped into at least three separate aqueous concentrated feeds and a diluent as disclosed for the compartmentalized serum-free cell culture perfusion medium according to the invention. Typically the cell culture and/or the reaction vessel of the bioreactor comprise mammalian cells upon addition of the at least three separate aqueous concentrated feeds and the diluent.

The method may comprise a step of sterilizing the concentrated feeds prior to storage and/or addition to the cell culture and/or the reaction vessel of the bioreactor, preferably by filter sterilization. The cell culture and/or the reaction vessel of the bioreactor comprise at least about 100 L serum-free cell culture perfusion medium, preferably at least about 1000 L serum-free cell culture perfusion medium.

Preferably the three concentrated feeds are added drop-wise through separate ports to the cell culture and/or the reaction vessel of the bioreactor. The in-vessel mixing and dilution of the at least three separate aqueous concentrated feeds allows 50-90%, preferably 60-90% lower prepared medium consumption over a culture period of 14 days compared to a serum-free cell culture perfusion medium mixed and diluted prior to addition to the bioreactor. The reduction in prepared medium consumption may be calculated using the formula provided above to calculate the maximal fold-concentration (nₘₐₓ X) upon mixing of the at least three separate aqueous concentrated feeds and calculating the percentage of the volume of the cumulative volume of the at least at least three separate aqueous concentrated feeds relative to the 1× serum free cell culture perfusion medium further comprising the diluent.

The separate addition of the at least three separate concentrated feeds from the diluent enables to control osmolality of the serum-free cell culture perfusion medium in the bioreactor. Osmolality serum-free cell culture perfusion medium in the bioreactor may also be controlled in case the diluent is premixed with one of the at least three separate aqueous concentrated feeds immediately before addition to the cell culture and/or the reaction vessel of the bioreactor.

The osmolality in the cell culture may be controlled using a constant concentrated feed perfusion rate and a varying diluent perfusion rate, resulting in a varying overall perfusion rate. A constant concentrated feed perfusion rate relates to a cumulative perfusion rate of the at least three separate aqueous concentrated feeds, more specifically the alkaline concentrated feed, the acidic concentrated feed and the near neutral concentrated feed. The overall perfusion rate is the cumulative perfusion rate of the at least three separate aqueous concentrated feeds and the diluent. Alternatively the osmolality in the cell culture may be controlled using a constant overall perfusion rate and a varying concentrated feed perfusion rate. This automatically results in a varying diluent perfusion rate. In another alternative the osmolality in the cell culture may be controlled using a constant diluent perfusion rate and a varying concentrated feed perfusion rate, resulting in a varying overall perfusion rate.

The at least three concentrated feeds are added at a fixed ratio (v/v/v) to each other depending on their fold-concentration to maintain the relative proportion of the medium components in the 1× serum-free cell culture perfusion medium. In one embodiment the ratio (v/v/v) of the alkaline concentrated feed to the acidic concentrated feed to the near neutral concentrated feed is a fixed ratio to provide the serum-free cell culture perfusion medium that is pH-adjusting to a neutral pH in the cell culture and/or the reaction vessel of the bioreactor; and the ratio (v/v) of the diluent to the cumulative volume of the at least three separate aqueous concentrated feeds added to the cell culture and/or the reaction vessel of the bioreactor to provide the serum-free cell culture perfusion medium that is pH-adjusting to a near neutral pH determines the osmolality and/or fold concentration of the serum-free cell culture perfusion medium in the cell culture and/or the reaction vessel of the bioreactor.

The osmolality in the cell culture may be increased using a constant concentrated feed perfusion rate and a decreased diluent perfusion rate, resulting in a decreased overall perfusion rate; or a constant overall perfusion rate and an increased concentrated feed perfusion rate with a decreased diluent perfusion rate; or a constant diluent perfusion rate and an increased concentrated feed perfusion rate, resulting in an increased overall perfusion rate; wherein the at least three concentrated feeds are added at a fixed ratio (v/v/v) to each other depending on their fold-concentration to maintain the relative proportion of the medium components in the 1× serum-free cell culture perfusion medium. In one embodiment. Preferably no further additive is added to the culture for increasing the osmolality.

In yet another aspect, the invention relates a serum-free cell culture perfusion medium obtainable by the method according to the invention.

### Cell Culture Methods

For the purposes of understanding it will be appreciated by the skilled practitioner that cell cultures and culturing runs for protein production can include at least three general types; namely, perfusion culture, batch culture and fed-batch culture. In a perfusion culture, for example, fresh culture medium supplement is provided to the cells during the culturing period, while old culture medium is removed daily and the product is harvested, for example, daily or continuously. In perfusion culture, perfusion medium can be added daily and can be added continuously, i.e., as a drip or infusion. For perfusion culturing, the cells can remain in culture as long as is desired, so long as the cells remain alive and the environmental and culturing conditions are maintained. Since the cells grow continuously, it is typically required to remove cells during the run in order to maintain a constant viable cell density, which is referred to as cell bleed. The cell bleed contains product in the culture medium removed with the cells, which is typically discarded and hence wasted. Thus, maintaining the viable cell density during production phase without or with only minimal cell bleeding is advantageous and increases the total yield per run.

In batch culture, cells are initially cultured in medium and this medium is not removed, replaced, or supplemented, i.e., the cells are not “fed” with new medium, during or before the end of the culturing run. The desired product is harvested at the end of the culturing run. Batch culture may also refer to the initial stage of fed-batch or perfusion culture. For perfusion culturing the mammalian cells may for example initially be cultured as batch culture before perfusion culture is stated.

For fed-batch cultures, the culturing run time is increased by supplementing the culture medium one or more times daily (or continuously) with fresh medium during the run, i.e., the cells are “fed” with new medium (“feeding medium”) during the culturing period. Fed-batch cultures can include the various feeding regimens and times as described above, for example, daily, every other day, every two days, etc., more than once per day, or less than once per day, and so on. Further, fed-batch cultures can be fed continuously with feeding medium. The desired product is then harvested at the end of the culturing/production run.

Mammalian cells may be cultured in perfusion culture. During heterologous protein production it is desirable to have a controlled system where cells are grown to a desired viable cell density and then the cells are switched to a growth-arrested, high productivity state where the cells use energy and substrates to produce the heterologous protein of interest rather than cell growth and cell division. Methods for accomplishing this goal, such as temperature shifts and amino acid starvation, are not always successful and can have undesirable effects on product quality. As described herein viable cell density during production phase can be maintained at a desirable level by performing a regular cell bleed. However, this results in discarding heterologous protein of interest. Cell growth-arrest during production phase results in a reduced need for a cell bleed and may even maintain cells in a more productive state.

In one aspect, a method of culturing mammalian cells expressing a heterologous protein in perfusion culture is provided, comprising: (a) inoculating a bioreactor with mammalian cells expressing a heterologous protein in a serum-free cell culture medium; (b) culturing the mammalian cells in a perfusion culture by continuously feeding the mammalian cells with a serum-free cell culture perfusion medium feed and removing spent media while keeping the cells in culture, wherein the serum-free cell culture medium perfusion feed is (i) a compartmentalized serum-free cell culture perfusion medium comprising the medium components subgrouped into at least three separate aqueous concentrated feeds and a diluent, wherein the first concentrated feed is an alkaline concentrated feed, the second concentrated feed is an acidic concentrated feed and the third concentrated feed is a near neutral concentrated feed; and wherein the compartmentalized serum-free cell culture perfusion medium is pH-adjusting to neutral pH upon mixing of the at least three separate aqueous concentrated feeds and the diluent in the resulting serum-free cell culture perfusion medium; and/or (ii) a serum-free cell culture perfusion medium obtained by the method according to the invention, and wherein the alkaline concentrated feed, the acidic concentrated feed and the near neutral concentrated feed of the compartmentalized serum-free cell culture perfusion medium feed are added separately to the cell culture and/or the reaction vessel of the bioreactor and wherein the diluent is added separately to the cell culture and/or the reaction vessel of the bioreactor or the diluent is premixed with one of the at least three separate aqueous concentrated feeds immediately before addition to the cell culture and/or the reaction vessel of the bioreactor.

In one embodiment the mammalian cells are initially cultured as a batch culture before perfusion culture is started. Typically, the serum-free cell culture medium in step (a) is a growth medium. Step (a) may further comprise culturing the mammalian cells in a growth medium and starting perfusion culture using said growth medium. Culturing the mammalian cells in a perfusion culture in step (b) comprises culturing the mammalian cells during production phase by perfusion with the serum-free cell culture medium according to the invention or the serum-free cell culture medium obtained by the method according to the invention until target cell density is reached; and further maintaining the mammalian cells during production phase at the target cell density by perfusion with the serum-free cell culture medium according to the invention or the serum-free cell culture medium obtained by the method according to the invention. The serum-free cell culture perfusion medium used in perfusion culture by continuously feeding the mammalian cells and removing spent media while keeping the cells in culture according to step (b) may be a production medium. The methods may further comprise a step of harvesting the heterologous protein from the cell culture.

The production phase typically starts before the target cell density is reached. The target cell density depends on the cell line and the maximal viable cell density of the cell line and is typically about 15-45% than the maximal viable cell density. Production phase may be started at a cell density of 10×10⁶ cells/ml to about 120×10⁶ cells/ml or even higher. Preferably production phase is initiated at a cell density of at least 10×10⁶ cells/ml, at least 20×10⁶ cells/ml, at least 30×10⁶ cells/ml, at least 40×10⁶ cells/ml or at least 50×10⁶ cells/ml. Typically the production phase is started when the culture reached 0.2±0.1 g/L_{bioreactor}/day or higher of heterologous protein in the permeate, which is the time when purification of the heterologous protein is started.

According to the methods of the invention, culturing the mammalian cells in step (a) may be limited to inoculating mammalian cells expressing a heterologous protein in a serum-free culture medium and hence does not need to but may include a culturing step prior to the start of perfusion and further does not need to but may include starting perfusion culture. Typically a growth medium is used in step (a), which is replaced by the medium according to the invention or obtained according to the method of the invention in step (b), also referred to as production medium. Further according to the methods of the invention, maintaining the mammalian cells during production phase by perfusion includes culturing the mammalian cells during production phase by perfusion at a substantially constant viable cell density at about the target viable cell density, wherein substantially constant viable cell density means a variation within 30%, preferably 20%, more preferably 10% of the viable cell density.

The invention also relates to a method of producing a heterologous protein comprising using the method of culturing mammalian cells expressing a heterologous protein in perfusion culture according to the invention. The person skilled in the art will understand that the methods according to the invention are in vitro culture methods.

In one embodiment, the serum-free cell culture perfusion medium may be chemically defined and/or hydrolysate-free. Preferably the serum-free cell culture perfusion medium is protein-free or protein-free except for recombinant insulin and/or insulin-like growth factor. Thus, the serum-free cell culture perfusion medium may be a protein-free medium or a protein-free medium comprising recombinant insulin and/or recombinant insulin-like growth factor. More preferably the serum-free perfusion medium is chemically defined and protein-free or protein-free except for recombinant insulin and/or insulin-like growth factor. This also applies to the serum-free culture medium used in step (a) of the methods according to the methods of the present invention.

The mammalian cells may initially be cultured as a batch culture before perfusion culture is started. Typically perfusion culture starts from day 0 to day 5, preferably from day 0 to day 4, more preferably from day 0 to day 3 of the culture. The perfusion rate increases after perfusion has started until a target viable cell density has been reached. The perfusion rate may for example increase from less or equal to 0.5 vessel volumes per day to about 5 vessel volumes per day, preferably from less or equal to 0.5 vessel volumes per day to about 2 vessel volumes per day.

As already explained above, the methods of the invention may further comprise a step that the cell density is maintained by cell bleeding at steady state. The cell density referred to in this context is the viable cell density, which may be determined by any method known in the art. For example the calculation governing the cell bleed rate may be based on maintaining the INCYTE™ viable cell density probe (HAMILTON® COMPANY) or FUTURA™ biomass capacitance probe value (ABER® instruments) which corresponded to the target VCD, or a daily cell and viability count can be taken off-line via any cell counting device, such as haemocytometer, VI-CELL XR™ (BECKMAN COULTER®), CEDEX HI-RES™ (ROCHE®), or VIACOUNT™ assay (EMD MILLIPORE® GUAVA EASYCYTE®). Using the methods of the present invention the cell bleeding may be eliminated or reduced compared to a control perfusion cell culture by increasing the osmolality, wherein a control perfusion cell culture is a perfusion cell culture that is cultured under the same conditions using the same serum-free perfusion medium without the osmolality being increased in the cell culture according to the invention. More specifically the cell bleeding may be reduced compared to a control perfusion cell culture, wherein a control perfusion cell culture is a perfusion cell culture that is cultured under the same conditions using the same serum-free perfusion medium without the osmolality being increased. A perfusion cell culture without cell bleeding may also be referred to as “dynamic perfusion culture” or “dynamic perfusion process”. Preferably a dynamic perfusion culture also comprises a high viable cell density, e.g., above 80×10⁶ cells/ml, above 100×10⁶ cells/ml, above 120×10⁶ cells/ml or even above 140×10⁶ cells/ml and/or a relatively short cultivation time of less than 30 days, preferably of 14-16 days.

In one embodiment the osmolality of the serum-free cell culture perfusion medium may be increased above the optimal osmolality level for growth, resulting in growth suppression of the mammalian cell at a target viable cell density, preferably wherein the osmolality level of the serum-free cell culture perfusion medium is increased gradually or stepwise starting at about half the target viable cell density. The target viable cell density may be about 30×10⁶ cells/ml or higher, about 60×10⁶ cells/ml or higher, about 80×10⁶ cells/ml, preferably about 100×10⁶ cells/ml or higher. The target viable cell density may be even as high as about 100×10⁶ cells/ml to about 200×10⁶ cells/ml, preferably about 120×10⁶ cells/ml to 150×10⁶ cells/ml. For cell lines with an inherent maximal viable cell density greater than 150×10⁶ cells/ml, cell growth typically needs to be inhibited to ensure adequate supply of oxygen, avoidance of excessive cell clumping (which can block cell retention devices), minimizing effects of waste metabolite accumulation, etc, although target viable cell densities of 200×10⁶ cells/ml have been achieved.

The osmolality in the cell culture may be controlled using a constant concentrated feed perfusion rate and a varying diluent perfusion rate, resulting in a varying overall perfusion rate. A constant concentrated feed perfusion rate relates to a cumulative or total perfusion rate of at least three separate aqueous concentrated feeds, more specifically the alkaline concentrated feed, the acidic concentrated feed and the near neutral concentrated feed. The concentrated feeds may, e.g., be fed at a constant total perfusion rate of 0.5 WD (e.g., 6× acidic feed at 0.33 WD, 25× alkaline and near neutral feed at 0.08 WD each). The overall perfusion rate is the cumulative perfusion rate of the at least three separate aqueous concentrated feeds and the diluent. Alternatively the osmolality in the cell culture may be controlled using a constant overall perfusion rate and a varying concentrated feed perfusion rate. This automatically results in a varying diluent perfusion rate. In another alternative the osmolality in the cell culture may be controlled using a constant diluent perfusion rate and a varying concentrated feed perfusion rate, resulting in a varying overall perfusion rate. The at least three concentrated feeds are added at a fixed ratio (v/v/v) to each other depending on their fold-concentration to maintain the relative proportion of the medium components in the 1× serum-free cell culture perfusion medium. In other words, the ratio (v/v/v) of the alkaline concentrated feed to the acidic concentrated feed to the near neutral concentrated feed is a fixed ratio (for each medium) to provide the serum-free cell culture perfusion medium that is pH-adjusting to a neutral pH in the cell culture and/or the reaction vessel of the bioreactor. Preferably the osmolality in the cell culture is controlled using a constant concentrated feed perfusion rate and a varying diluent perfusion rate, resulting in a varying overall perfusion rate.

The osmolality (and the fold-concentration of the serum-free cell culture perfusion medium) in the cell culture may be increased using a constant concentrated feed perfusion rate and a decreased diluent perfusion rate, resulting in a decreased overall perfusion rate; or a constant overall perfusion rate and an increased concentrated feed perfusion rate with a decreased diluent perfusion rate; or a constant diluent perfusion rate and an increased concentrated feed perfusion rate, resulting in an increased overall perfusion rate; wherein the at least three concentrated feeds are added at a fixed ratio (v/v/v) to each other depending on their fold-concentration to maintain the relative proportion of the medium components in the 1× serum-free cell culture perfusion medium. Preferably no further additive (such as NaCl) is added to the culture for increasing the osmolality. Preferably the osmolality in the cell culture is increased using a constant concentrated feed perfusion rate and a decreased diluent perfusion rate, resulting in a decreased overall perfusion rate. In a preferred embodiment no further additive is added to the culture for increasing the osmolality.

Increasing the ratio (v/v) of the diluent to the cumulative volume of the at least three separate aqueous concentrated feeds added to the cell culture and/or the reaction vessel of the bioreactor to provide the serum-free cell culture perfusion medium that is pH-adjusting to a near neutral pH determines the osmolality and also the fold-concentration of the resulting serum-free cell culture perfusion medium in the cell culture and/or the reaction vessel of the bioreactor. The fold-concentration can be anything from 0.1× to the maximal fold-concentration of the serum-free cell culture medium, which can be calculated as explained above. Using concentrated feeds allows to adjust the fold-concentration of the serum-free cell culture medium in the cell culture and/or bioreactor and hence in addition to allowing regulating growth suppression by increasing the osmolality, it allows to increase the nutrient content in the medium by increasing the fold-concentration of the serum-free cell culture medium. This allows maintenance of higher viable cell densities at a similar or only moderately increased perfusion rate and consequently at a reduced cell specific perfusion rate. The term “fold-concentrated” refers to a concentrate (n>1) or a dilution (n>1) of a 1× serum-free cell culture perfusion medium, wherein a 1× serum-free cell culture perfusion medium is the originally prepared or designed serum-free cell culture perfusion medium formulation.

The ratio (v/v) of the diluent to the cumulative volume of the at least three separate aqueous concentrated feeds added to the cell culture and/or the reaction vessel of the bioreactor to provide the resulting serum-free cell culture perfusion medium that is pH-adjusting to a near neutral pH also determines the fold-concentration (overall nutrient content) of the serum-free cell culture perfusion medium in the cell culture and/or the reaction vessel of the bioreactor. Thus, the advantage of using concentrated feeds is that the fold-concentration of the medium may be adapted to the viable cell concentration and the nutritive needs (maintain nutritive balance). Osmolality may be used as a surrogate to estimate nutritive balance in and out of the system. Thus, an osmo balance may be used to calculate adjustment of the cumulative volume of the concentrated feeds (at their fixed ratio to each other) and diluent feed rates to achieve a desirable residual osmolality and nutritive level.

Any feeding strategy must take into consideration osmolality added by any other feeds, such as glucose or basic titrant. Osmolality control scheme selection is cell line dependent and depends of the sensitivity of each cell line to osmolality and waste product accumulation. The lowest possible perfusion rate is preferred. The rate of feeds may be determined based on the known osmolality of the concentrated feeds and the assumed cell specific osmolality consumption rate, which is calculated on a day-to-day basis. The osmo balance for the daily osmolality consumption may be calculated according to the following equation:

osmo input−osmo output=osmo consumption,

where osmo input is the osmolality of media concentrate feeds and diluent perfusing into the bioreactor, osmo output is the residual osmolality of the bioreactor supernatant, and osmo consumption is the difference in osmolality between the input and output. This daily osmo consumption is then normalized to the number of cells in the culture, for a daily per cell osmolality consumption. This daily consumption rate per cell (or cell-specific osmo consumption rate, CSOCR) is then multiplied by the following day's predicted VCD to predict the following day's osmo consumption. This consumption rate along with the desired osmo output can be used to calculate the required osmo input for the following day. The perfusion rate of the diluent and/or the concentrated feeds are then adjusted to match the osmo input target.

The optimal osmolality level for growth in a cell culture is cell line dependent and may be between about 280 mOsm to about 390 mOsm, more preferably between 280 to less than about 350 mOsm (mOsmol/kg water). Some cell lines may still grow optimally at an osmolality above 390 mOsm. The optimal osmolarity level for growth of a mammalian cell in a cell culture depends on the mammalian cell used and possibly also the culture conditions. The optimal osmolality level for growth of a mammalian cell may be easily determined by determining the viable cell density and viability at different osmolalities. The optimal osmolality level is cell density independent and but is preferably determined at about target viable cell density. The osmolality should be maintained at a level optimal for growth at least until about half the target viable cell density is reached.

Once the target viable cell density is reached the osmolality may be increased to suppress cell growth, such as increased by about 10-70%, about 10-60% or about 10-50% of the optimal osmolality level for growth of the mammalian cell. The osmolality should be increased gradually or stepwise, preferably starting at about half the target viable cell density (i.e., approximately one population doubling away from the target viable cell density), more preferably to about 10-70%, about 10-60% or about 10-50% of the optimal osmolality level for growth. In one embodiment the osmolality is increased to about 350 mOsm or higher, preferably to about 380 mOsm or higher, to about 400 mOsm or higher, to about 420 mOsm or higher or to about 450 mOsm or higher The osmolality is increased to a level that suppresses cell growth of the mammalian cell without being cytotoxic to the mammalian cell. The osmolality may be increased to and maintained at an osmolality level that suppresses cell growth of the mammalian cell, preferably at about the target viable cell density, wherein the osmolality level that suppresses cell growth of the mammalian cell may be about 350 mOsm or higher, or 380 mOsm or higher. However, it is important that cell viability of the mammalian cell is not substantially affected. For most cell lines osmolality levels start to become cytotoxic above about 400 mOsm, but for individual cell lines osmolality levels may be increased to 450 mOsm without affecting cytotoxicity. The increase in osmolality to physiologically stressful levels inhibits cell growth. The osmolality that inhibits cell growth of a mammalian cell in a cell culture depends on the mammalian cell used. The osmolality that inhibits cell growth of a specific mammalian cell in a cell culture without reaching cytotoxic levels may be easily determined by measuring the viable cell density and viability at different osmolalities. Preferably the increased osmolality results in maintaining the cells during production phase at about target viable cell density without affecting viability. Thus, increasing the osmolality reduces or eliminates the need for cell bleeding during production phase. By increasing the osmolality in the cell culture, cell growth may be suppressed to maintain a sustainable viable cell density without cell bleeding, particularly a high viable cell density without cell bleeding, such as <100×10⁶ cells/ml, preferably <120×10⁶ cells/ml, which may also be referred to as a dynamic perfusion culture.

By increasing the osmolality in the cell culture, the yield of the heterologous protein produced in the cell culture may be increased by at least about 5%, at least about 10% at least about 25%, at least about 50%, at least about 75%, at least about 100 percent, or about 5-50%, preferably about 10 to 100% relative to the yield in a control cell culture, wherein the osmolality is not increased. Preferable the yield is determined for a part or the entire culture period.

By using the serum-free cell culture medium according to the invention or the serum-free cell culture medium obtained by the method according to the invention and optionally further increasing the osmolality in the cell culture, the cell specific perfusion rate (pl/cell/day) is reduced by at least about 25%, at least about 30% or at least about 50%, relative to the cell specific perfusion rate of a 1× serum-free cell culture medium.

In one embodiment of the methods of the present invention the cell culture and/or the reaction vessel of the bioreactor comprise at least about 100 L serum-free cell culture perfusion medium, preferably at least about 1000 L serum-free cell culture perfusion medium. Preferably the cell culture has a volume of at least about 100 L and/or the bioreactor has a volume of at least about 100 L. More preferably the cell culture has a volume of at least about 1000 L and/or the bioreactor has a volume of at least about 1000 L. Although the serum-free cell culture perfusion medium used in or prepared by the methods of the invention is a complete serum-free cell culture perfusion medium the culture may further be supplemented. Suitable supplements that may be added separately to the cell culture are, without being limited thereto anti-foaming agents, base, glucose and/or glutamine.

The heterologous protein may be any protein, preferably it is a therapeutic protein, such as an antibody, or a therapeutically effective fragment thereof, a fusion protein or a cytokine or any of the heterologous proteins described herein. The antibody may be a monoclonal antibody, a bispecific antibody, a multimeric antibody or a fragment thereof.

### Bioreactors

The serum-free cell culture perfusion medium may be utilized in any type of cell culture system, type, or format, suitable for continuous perfusion.

Any cell perfusion bioreactor and cell retention device may be used for perfusion culture. The bioreactors used for perfusion are not very different from those used for batch/fed-batch cultures, except that they are more compact in size and are connected to a cell retention device. The methods for retaining cells inside the bioreactor are primarily determined by whether the cells are growing attached to surfaces or growing in either single cell suspension or cell aggregates. While most mammalian cells historically were grown attached to a surface or a matrix (heterogeneous cultures), efforts have been made to adapt many industrial mammalian cell lines to grow in suspension (homogenous cultures), mainly because suspension cultures are easier to scale-up. Thus, the cells used in the methods of the invention are preferably grown in suspension. Without being limited thereto, exemplary retention systems for cells grown in suspension are spin filter, external filtration such as tangential flow filtration (TFF), alternating tangential flow (ATF) system, cell sedimentation (vertical sedimentation and inclined sedimentation), centrifugation, ultrasonic separation and hydrocyclones. Perfusion systems can be categorized into two categories, filtration based systems, such as spin filters, external filtration and ATF, and open perfusion systems, such as gravitational settlers, centrifuges, ultrasonic separation devices and hydroclones. Filtration-based systems show a high degree of cell retention and it does not change with the flow rate. However, the filters may clog and hence the cultivation run is limited in length or the filters need to be exchanged. An example for an ATF system is the XCELL™ ATF system from REPLIGEN™ and an example for a TFF system is the TFF system from LEVITRONIX® using a centrifugal pump. A cross-flow filter, such as a hollow fiber (HF) or a flat plate filter may be used with ATF and TFF systems. Specifically a hollow fiber, made of modified polyethersulfone (mPES), polyethersulfone (PES), or polysulfone (PE), can be used with ATF and TFF systems. Pore sizes of the HF can range from several hundred kDa to 15 μM. Open perfusion systems do not clog and hence could at least theoretically be operated indefinitely. However, the degree of cell retention is reduced at higher perfusion rates. Currently there are three systems that can be used at industrial scale, alternating tangential filters (ATF), gravitational (particularly inclined settlers) and centrifuges. Cell retention devices suitable for heterogeneous or homogenous cultures are described in more detail by Kompala and Ozturk (Cell Culture Technology for Pharmaceutical and Cell-Based Therapies, (2006), Taylor & Francis Group, LLC, pages 387-416), which is incorporated herein by reference. The perfusion culture is not a true steady state process, with the total and viable cell concentration reaching a steady state only when a cell bleed stream is removed from the bioreactor.

Physical parameters such as pH, dissolved oxygen and temperature in a perfusion bioreactor should be monitored on-line and controlled in real time. Determination of cell density, viability, metabolite, and product concentrations may be performed using off-line or on-line sampling. When the perfusion operation starts with continuous harvest and feeding the perfusion rate typically refers to the harvest flow rate, which may be manually set to a desired value. For example, a weight control for the bioreactor may activate the feed pump so that a constant volume in the bioreactor can be maintained. Alternatively, a level control can be achieved by pumping out culture volume above a predetermined level. The perfusion rate in the bioreactor must be adjusted to deliver sufficient nutrients to the cells.

Perfusion rate may be controlled, e.g., using cell density measurements, pH measurements, oxygen consumption or metabolite measurements. Cell density is the most important measurement used for perfusion rate adjustments. Depending on how the cell density measurements are conducted, perfusion rates can be adjusted daily or in real time. Several on-line probes have been developed for the estimation of cell density and are known to the person skilled in the art, such as a capacitance probe, e.g., an INCYTE™ viable cell density probe (HAMILTON® COMPANY) or FUTURA™ biomass capacitance probe value (ABER® instruments). These cell density probes can also be used to control the cell density at a desired set point by removing excess cells from the bioreactor, i.e., the cell bleed. Thus, the cell bleed is determined by the specific growth rate of the mammalian cells in culture. The cell bleed is typically not harvested and therefore considered as waste.

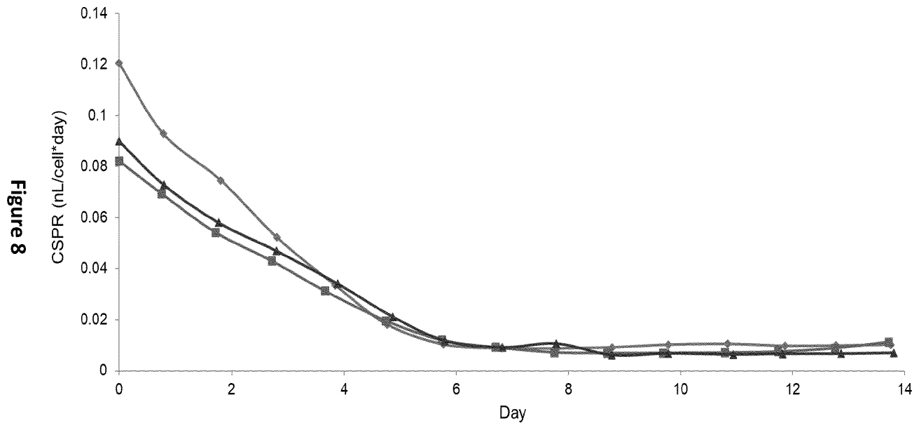

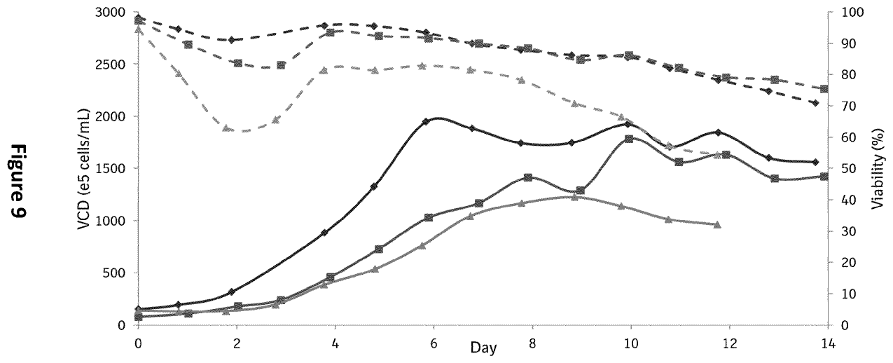

The methods of the present invention further comprise harvesting the heterologous protein from the perfusion cell culture. The invention contemplates any suitable method for harvesting and purifying the protein of interest. The harvesting may also occur intermittently throughout the cell culture life cycle, or at the end of the cell culture. Harvesting is preferably done continuously from the permeate, which is the supernatant produced after cells have been recovered by a cell retention device. Due to the lower product residence time of the product proteins in the cell culture inside the perfusion bioreactor compared to fed-batch, the exposure to proteases, sialidases and other degrading proteins is minimized, which may result in better product quality of heterologous proteins produced in perfusion culture. Preferably the harvested product is purified using iSKID as described in U.S. provisional application 62/827,504, particularly FIG. 6 thereof. iSKID are integrated skids that bring together multiple-unit operations in a highly automated fashion and can take on complete continuous automated manufacturing.

### Expression Products

The heterologous protein produced by the methods and uses of the present invention may be any secreted protein, preferably it is a therapeutic protein. Since most therapeutic proteins are recombinant therapeutic proteins, it is most preferably a recombinant therapeutic protein. Examples for therapeutic proteins are without being limited thereto antibodies, fusion proteins, cytokines and growth factor.

The therapeutic protein produced in the mammalian cells according to the methods of the invention includes, but is not limited to an antibodies or a fusion protein, such as a Fc-fusion proteins. Other secreted recombinant therapeutic proteins can be for example enzymes, cytokines, lymphokines, adhesion molecules, receptors and derivatives or fragments thereof, and any other polypeptides and scaffolds that can serve as agonists or antagonists and/or have therapeutic or diagnostic use.

Other recombinant proteins of interest are for example, without being limited thereto: insulin, insulin-like growth factor, hGH, tPA, cytokines, such as interleukins (IL), e.g. IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, interferon (IFN) alpha, IFN beta, IFN gamma, IFN omega or IFN tau, tumor necrosis factor (TNF), such as TNF alpha and TNF beta, TNF gamma, TRAIL; G-CSF, GM-CSF, M-CSF, MCP-1, and VEGF. Also included is the production of erythropoietin or any other hormone growth factors and any other polypeptides that can serve as agonists or antagonists and/or have therapeutic or diagnostic use.

A preferred therapeutic protein is an antibody or a fragment or derivative thereof, more preferably an IgG1 antibody. Thus, the invention can be advantageously used for production of antibodies such as monoclonal antibodies, multi-specific antibodies, or fragments thereof, preferably of monoclonal antibodies, bi-specific antibodies or fragments thereof. Exemplary antibodies within the scope of the present invention include but are not limited to anti-CD2, anti-CD3, anti-CD20, anti-CD22, anti-CD30, anti-CD33, anti-CD37, anti-CD40, anti-CD44, anti-CD44v6, anti-CD49d, anti-CD52, anti-EGFR1 (HER1), anti-EGFR2 (HER2), anti-GD3, anti-IGF, anti-VEGF, anti-TNFalpha, anti-IL2, anti-IL-SR or anti-IgE antibodies, and are preferably selected from the group consisting of anti-CD20, anti-CD33, anti-CD37, anti-CD40, anti-CD44, anti-CD52, anti-HER2/neu (erbB2), anti-EGFR, anti-IGF, anti-VEGF, anti-TNFalpha, anti-IL2 and anti-IgE antibodies.

Antibody fragments include e.g. “Fab fragments” (Fragment antigen-binding=Fab). Fab fragments consist of the variable regions of both chains, which are held together by the adjacent constant region. These may be formed by protease digestion, e.g. with papain, from conventional antibodies, but similarly Fab fragments may also be produced by genetic engineering. Further antibody fragments include F(ab′)2 fragments, which may be prepared by proteolytic cleavage with pepsin.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins are known to the person skilled in the art.

Preferred therapeutic antibodies according to the invention are bispecific antibodies. Bispecific antibodies typically combine antigen-binding specificities for target cells (e.g., malignant B cells) and effector cells (e.g., T cells, NK cells or macrophages) in one molecule. Exemplary bispecific antibodies, without being limited thereto are diabodies, BiTE (Bi-specific T-cell Engager) formats and DART (Dual-Affinity Re-Targeting) formats. The diabody format separates cognate variable domains of heavy and light chains of the two antigen binding specificities on two separate polypeptide chains, with the two polypeptide chains being associated non-covalently. The DART format is based on the diabody format, but it provides additional stabilization through a C-terminal disulfide bridge.

Another preferred therapeutic protein is a fusion protein, such as an Fc-fusion protein. Thus, the invention can be advantageously used for production of fusion proteins, such as Fc-fusion proteins. Furthermore, the method of increasing protein producing according to the invention can be advantageously used for production of fusion proteins, such as Fc-fusion proteins.

The effector part of the fusion protein can be the complete sequence or any part of the sequence of a natural or modified heterologous protein or a composition of complete sequences or any part of the sequence of a natural or modified heterologous protein. The immunoglobulin constant domain sequences may be obtained from any immunoglobulin subtypes, such as IgG1, IgG2, IgG3, IgG4, IgA1 or IgA2 subtypes or classes such as IgA, IgE, IgD or IgM. Preferentially they are derived from human immunoglobulin, more preferred from human IgG and even more preferred from human IgG1 and IgG2 Non-limiting examples of Fc-fusion proteins are MCP1-Fc, ICAM-Fc, EPO-Fc and scFv fragments or the like coupled to the CH2 domain of the heavy chain immunoglobulin constant region comprising the N-linked glycosylation site. Fc-fusion proteins can be constructed by genetic engineering approaches by introducing the CH2 domain of the heavy chain immunoglobulin constant region comprising the N-linked glycosylation site into another expression construct comprising for example other immunoglobulin domains, enzymatically active protein portions, or effector domains. Thus, an Fc-fusion protein according to the present invention comprises also a single chain Fv fragment linked to the CH2 domain of the heavy chain immunoglobulin constant region comprising e.g. the N-linked glycosylation site.

### Recovery of and Formulation of Expression Products

In a further aspect a method of producing a therapeutic protein is provided using the methods of the invention and optionally further comprising a step of purifying and formulating the therapeutic protein into a pharmaceutically acceptable formulation.

The therapeutic protein, especially the antibody, antibody fragment or Fc-fusion protein is preferably recovered/isolated from the culture medium as a secreted polypeptide. It is necessary to purify the therapeutic protein from other recombinant proteins and host cell proteins to obtain substantially homogenous preparations of the therapeutic protein. As a first step, cells and/or particulate cell debris are removed from the culture medium. Further, the therapeutic protein is purified from contaminant soluble proteins, polypeptides and nucleic acids, for example, by fractionation on immunoaffinity or ion-exchange columns, ethanol precipitation, reverse phase HPLC, Sephadex chromatography, and chromatography on silica or on a cation exchange resin such as DEAE. Methods for purifying a heterologous protein expressed by mammalian cells are known in the art.

### Expression Vectors

In one embodiment the heterologous protein expressed using the methods of the invention is encoded by one or more expression cassette(s) comprising a heterologous polynucleotide coding for the heterologous protein. The heterologous protein may be placed under the control of an amplifiable genetic selection marker, such as dihydrofolate reductase (DHFR), glutamine synthetase (GS). The amplifiable selection marker gene can be on the same expression vector as the heterologous protein expression cassette. Alternatively, the amplifiable selection marker gene and the heterologous protein expression cassette can be on different expression vectors, but integrate in close proximity into the host cell's genome. Two or more vectors that are co-transfected simultaneously, for example, often integrate in close proximity into the host cell's genome. Amplification of the genetic region containing the secreted therapeutic protein expression cassette is then mediated by adding the amplification agent (e.g., MTX for DHFR or MSX for GS) into the cultivation medium.

Sufficiently high stable levels of a heterologous protein expressed by a mammalian cell may also be achieved, e.g., by cloning multiple copies of the heterologous protein encoding-polynucleotide into an expression vector. Cloning multiple copies of the heterologous protein-encoding polynucleotide into an expression vector and amplifying the heterologous protein expression cassette as described above may further be combined.

### Mammalian Cell Lines

Mammalian cells as used herein are mammalian cells lines suitable for the production of a secreted recombinant therapeutic protein and may hence also be referred to as “host cells”. Preferred mammalian cells according to the invention are rodent cells such as hamster cells. The mammalian cells are isolated cells or cell lines. The mammalian cells are preferably transformed and/or immortalized cell lines. They are adapted to serial passages in cell culture and do not include primary non-transformed cells or cells that are part of an organ structure. Preferred mammalian cells are BHK21, BHK TK−, Jurkat cells, 293 cells, HeLa cells, CV-1 cells, 3T3 cells, CHO, CHO-K1, CHO-DXB11 (also referred to as CHO-DUKX or DuxB11), a CHO-S cell and CHO-DG44 cells or the derivatives/progenies of any of such cell line. Particularly preferred are CHO cells, such as CHO-DG44, CHO-K1 and BHK21, and even more preferred are CHO-DG44 and CHO-K1 cells. Most preferred are CHO-DG44 cells. Glutamine synthetase (GS)-deficient derivatives of the mammalian cell, particularly of the CHO-DG44 and CHO-K1 cell are also encompassed. In one embodiment of the invention the mammalian cell is a Chinese hamster ovary (CHO) cell, preferably a CHO-DG44 cell, a CHO-K1 cell, a CHO DXB11 cell, a CHO-S cell, a CHO GS deficient cell or a derivative thereof.

The mammalian cell may further comprise one or more expression cassette(s) encoding a heterologous protein, such as a therapeutic protein, preferably a recombinant secreted therapeutic protein. The host cells may also be murine cells such as murine myeloma cells, such as NS0 and Sp2/0 cells or the derivatives/progenies of any of such cell line. Non-limiting examples of mammalian cells which can be used in the meaning of this invention are also summarized in Table 1. However, derivatives/progenies of those cells, other mammalian cells, including but not limited to human, mice, rat, monkey, and rodent cell lines, can also be used in the present invention, particularly for the production of biopharmaceutical proteins.

TABLE 1
Mammalian production cell lines

| Cell line | Order Number |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHO-DUKX | ATCC CRL-9096 |
| (= CHO duk⁻, CHO/dhfr^{-,}, | |
| CHO-DXB11) | |
| CHO-DUKX 5A-HS-MYC | ATCCCRL-9010 |
| CHO-DG44 | Urlaub G, et al., 1983. Cell. |
| | 33: 405-412. |
| CHO Pro-5 | ATCC CRL-1781 |
| CHO-S | Life Technologies A1136401; |
| | CHO-S is derived from CHO |
| | variant Tobey etal. 1962 |
| V79 | ATCC CCC-93 |
| B14AF28-G3 | ATCC CCL-14 |
| HEK293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |
| CAP¹ | Wölfel J, et al., 2011. BMC Proc. |
| | 5(Suppl 8): P133. |
| PER.C6 ® | Pau et al., 2001. Vaccines. 19: |
| | 2716-2721. |
| H4-II-E | ATCC CRL-1548 |
| | ECACC No.87031301 |
| | Reuber, 1961. J. Natl. Cancer Inst. |
| | 26: 891-899. |
| | Pitot HC, et al., 1964. Natl. |
| | Cancer Inst. Monogr. 13: 229-245. |
| H4-II-E-C3 | ATCC CRL-1600 |
| H4TG | ATCC CRL-1578 |
| H4-II-E | DSM ACC3129 |
| H4-II-Es | DSM ACC3130 |

¹CAP (CEVEC's Amniocyte Production) cells are an immortalized cell line based on primary human amniocytes. They were generated by transfection of these primary cells with a vector containing the functions E1 and pIX of adenovirus 5. CAP cells allow for competitive stable production of recombinant proteins with excellent biologic activity and therapeutic efficacy as a result of authentic human posttranslational modification.

Mammalian cells are most preferred, when being established, adapted, and completely cultivated under serum free conditions, and optionally in media, which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, Calif.), CHO-S-Invitrogen), serum-free CHO Medium (Sigma), and protein-free CHO Medium (Sigma) are exemplary appropriate nutrient solutions. Any of the media may be supplemented as necessary with a variety of compounds, non-limiting examples of which are recombinant hormones and/or other recombinant growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics and trace elements. Any other necessary supplements may also be included at appropriate concentrations that would be known to those skilled in the art. For the growth and selection of genetically modified cells expressing a selectable gene a suitable selection agent is added to the culture medium.

In view of the above, it will be appreciated that the invention also encompasses the following items:

Item 1 provides a compartmentalized serum-free cell culture perfusion medium comprising the medium components subgrouped into at least three separate aqueous concentrated feeds and a diluent, wherein the first concentrated feed is an alkaline concentrated feed, the second concentrated feed is an acidic concentrated feed and the third concentrated feed is a near neutral concentrated feed; wherein
the compartmentalized serum-free cell culture perfusion medium is pH-adjusting to neutral pH upon mixing of the at least three separate aqueous concentrated feeds and the diluent in the resulting serum-free cell culture perfusion medium.
Item 2 specifies the compartmentalized serum-free cell culture perfusion medium of item 1, wherein the resulting serum-free cell culture perfusion medium has a pH of between 6.7 and 7.5, between 6.9 and 7.4, preferably between 6.9 and 7.2, upon mixing of the at least three separate aqueous concentrated feeds and the diluent.
Item 3 specifies the compartmentalized serum-free cell culture perfusion medium of item 1 or 2, wherein the diluent is sterile water.
Item 4 specifies the compartmentalized serum-free cell culture perfusion medium of any one of items 1 to 3, wherein the compartmentalized serum-free cell culture perfusion medium is for
(a) separate addition of the alkaline concentrated feed, the acidic concentrated feed and the near neutral concentrated feed to a cell culture and/or a reaction vessel of a bioreactor;
(b) direct addition of the alkaline concentrated feed, the acidic concentrated feed and the near neutral concentrated feed to a cell culture and/or a reaction vessel of a bioreactor without prior pre-mixing; and/or
(c) direct mixing of the at least three separate aqueous concentrated feeds in a cell culture and/or a reaction vessel of a bioreactor.
   Item 5 specifies the compartmentalized serum-free cell culture perfusion medium of any one of the preceding items, wherein the alkaline concentrated feed is a 2× to 80× concentrated feed, the acidic concentrated feed is a 2× to 40× concentrated feed and the near neutral concentrated feed is a 2× to 50× concentrated feed.
   Item 6 specifies the compartmentalized serum-free cell culture perfusion medium of item 5, wherein
(a) the alkaline concentrated feed is a 20× to 40× concentrated feed, the acidic concentrated feed is a 4× to 20× concentrated feed and the near neutral concentrated feed is a 10× to 40× concentrated feed;
(b) the alkaline concentrated feed is a 20× to 30× concentrated feed, the acidic concentrated feed is a 5× to 12× concentrated feed and the near neutral concentrated feed is a 20× to 30× concentrated feed; and/or
(c) the alkaline concentrated feed is a 25× concentrated feed, the acidic concentrated feed is a 6× to 10× concentrated feed and the near neutral concentrated feed is a 25× concentrated feed.
   Item 7 specifies the compartmentalized serum-free cell culture perfusion medium of any one of the preceding items, wherein the near neutral concentrated feed has a pH of 6.5-8.5.
   Item 8 specifies the compartmentalized serum-free cell culture perfusion medium of any one of the preceding items, wherein the alkaline concentrated feed has a pH of 9 or higher, the acidic concentrated feed has a pH of 5 or lower and the near neutral concentrated feed has a pH of 7 to 8.5.
   Item 9 specifies the compartmentalized serum-free cell culture perfusion medium of item 8, wherein
(a) the alkaline concentrated feed has a pH of 9 to 11, the acidic concentrated feed has a pH of 2 to 5 and the near neutral concentrated feed has a pH of 7 to 8.5;
(b) the alkaline concentrated feed has a pH of 9.8 to 10.8, the acidic concentrated feed has a pH of 3.6 to 4.8 and the near neutral concentrated feed has a pH of 7 to 8.5; or
(c) the alkaline concentrated feed has a pH of 9.8 to 10.5, the acidic concentrated feed has a pH of 3.8 to 4.5 and the near neutral concentrated feed has a pH of 7.5 to 8.5.
   Item 10 specifies the compartmentalized serum-free cell culture perfusion medium of any one of the preceding items, wherein the resulting serum-free cell culture perfusion medium is (a) a chemically defined medium, (b) a hydrolysate-free medium, and/or (c) a protein-free medium or a protein-free medium comprising recombinant insulin and/or recombinant insulin-like growth factor.
   Item 11 specifies the compartmentalized serum-free cell culture perfusion medium of any one of the preceding items, wherein the resulting serum-free cell culture perfusion medium is a production medium.
   Item 12 specifies the compartmentalized serum-free cell culture perfusion medium of any one of the preceding items, wherein
(a) the ratio (v/v/v) of the alkaline concentrated feed to the acidic concentrated feed to the near neutral concentrated feed is a fixed ratio to provide the resulting serum-free cell culture perfusion medium that is pH-adjusting to a neutral pH; and
(b) the ratio (v/v) of the diluent to the cumulative volume of the at least three separate aqueous concentrated feeds in the resulting serum-free cell culture perfusion medium that is pH-adjusting to a neutral pH determines the osmolality of the serum-free cell culture perfusion medium.
   Item 13 specifies the compartmentalized serum-free cell culture perfusion medium of any one of the preceding items, wherein the acidic concentrated feed comprises trace elements, trace metals, inorganic salts, chelators, polyamines, and regulatory hormones.
   Item 14 specifies the compartmentalized serum-free cell culture perfusion medium of any one of the preceding items, wherein the acidic concentrated feed and/or the near neutral concentrated feed comprise surfactants, anti-oxidants, and carbon sources.
   Item 15 specifies the compartmentalized serum-free cell culture perfusion medium of any one of the preceding items, wherein the alkaline concentrated feed comprises amino acids with maximum solubility at alkaline pH of 9 or higher, preferably comprising at least aspartic acid, histidine and tyrosine, and optionally cysteine and/or cystine and/or folic acid.
   Item 16 specifies the compartmentalized serum-free cell culture perfusion medium of item 15, wherein the remaining amino acids are in the acidic and/or near neutral concentrated feed, preferably in the acidic concentrated feed.
   Item 17 specifies the compartmentalized serum-free cell culture perfusion medium of any one of the preceding items wherein the vitamins and the metals are in separate feeds, preferably vitamins are in the near neutral feed and metals are in the acidic feed.
   Item 18 specifies the compartmentalized serum-free cell culture perfusion medium of item 17 wherein vitamins poorly soluble in aqueous solutions, such as choline chloride, are present in the neutral feed and the acidic feed.
   Item 19 specifies an alkaline aqueous concentrated feed for combination with an acidic aqueous concentrated feed, a near neutral aqueous concentrated feed and a diluent to form a serum-free cell culture perfusion medium, wherein the pH of the resulting serum-free cell culture perfusion medium is automatically adjusted to a neutral pH.
   Item 20 specifies an acidic aqueous concentrated feed for combination with an alkaline aqueous concentrated feed, a near neutral aqueous concentrated feed and a diluent to form a serum-free cell culture perfusion medium, wherein the pH of the resulting serum-free cell culture perfusion medium is automatically adjusted to a neutral pH.
   Item 21 specifies a near neutral aqueous concentrated feed for combination with an alkaline aqueous concentrated feed, an acidic aqueous concentrated feed and a diluent to form a serum-free cell culture perfusion medium, wherein the pH of the resulting serum-free cell culture perfusion medium is automatically adjusted to a neutral pH.
   Item 22 specifies a method of preparing a serum-free cell culture perfusion medium comprising
   (a) providing the components of a cell culture media in at least three subgroups of components based on solubility at alkaline, acidic and neutral pH,
   (b) dissolving
      (i) the subgroup of components soluble at alkaline pH in an alkaline aqueous solution to form an alkaline concentrated feed;
      (ii) the subgroup of components soluble at acidic pH in an acidic aqueous solution to form an acidic concentrated feed; and
      (iii) the subgroup of components soluble at neutral pH in a neutral aqueous solution to form a near neutral concentrated feed;
   (c) optionally storing the prepared alkaline concentrated feed, acidic concentrated feed and near neutral concentrated feed in separate containers; and
   (d) adding the prepared alkaline concentrated feed, acidic concentrated feed and near neutral concentrated feed and the diluent to the cell culture and/or the reaction vessel of the bioreactor, wherein
      (i) the alkaline concentrated feed, the acidic concentrated feed and the near neutral concentrated feed are added separately to the cell culture and/or the reaction vessel of the bioreactor; and
      (ii) the diluent is added separately to the cell culture and/or the reaction vessel of the bioreactor or the diluent is premixed with one of the at least three separate aqueous concentrated feeds immediately before addition to the cell culture and/or the reaction vessel of the bioreactor;
         wherein the pH of the resulting serum-free cell culture perfusion medium is automatically pH adjusted to a neutral pH upon mixing of the at least three separate aqueous concentrated feeds and the diluent.
         Item 23 specifies the method of item 22, wherein the pH of the pH adjusted serum-free cell culture perfusion medium is between 6.7 and 7.5, between 6.9 and 7.4, preferably between 6.9 and 7.2, upon mixing of the at least three separate aqueous concentrated feeds and the diluent.
         Item 24 specifies the method of item 22 or 23, wherein the diluent is sterile water.
         Item 25 specifies the method of any one of items 22-24, wherein the three concentrated feeds are added drop-wise through separate ports to the cell culture and/or the reaction vessel of the bioreactor.
         Item 26 specifies the method of any one of items 22-25, wherein the in-vessel mixing and dilution of the at least three separate aqueous concentrated feeds allows 50-90%, preferably 60-90% lower prepared medium consumption over a culture period of 14 days compared to a serum-free cell culture perfusion medium mixed and diluted prior to addition to the bioreactor.
         Item 27 specifies the method of any one of items 22-26, wherein the cell culture and/or the reaction vessel of the bioreactor comprise mammalian cells.
         Item 28 specifies the method of any one of items 22-27, further comprising a step of sterilizing the concentrated feeds prior to storage and/or addition to the cell culture and/or the reaction vessel of the bioreactor.
         Item 29 specifies the method of any one of items 22-28, wherein the alkaline concentrated feed is a 2× to 80× concentrated feed, wherein the acidic concentrated feed is a 2× to 40× concentrated feed and the near neutral concentrated feed is a 2× to 50× concentrated feed.
         Item 30 specifies the method of item 29, wherein
   (a) the alkaline concentrated feed is a 20× to 40× concentrated feed, the acidic concentrated feed is a 4× to 20× concentrated feed and the near neutral concentrated feed is a 10× to 40× concentrated feed;
   (b) the alkaline concentrated feed is a 20× to 30× concentrated feed, the acidic concentrated feed is a 5× to 12× concentrated feed and the near neutral concentrated feed is a 20× to 30× concentrated feed; and/or
   (c) the alkaline feed is a 25× concentrated feed, the acidic concentrated feed is a 6× to 10× concentrated feed and the near neutral concentrated feed is a 25× concentrated feed.
      Item 31 specifies the method of any one of items 22-30, wherein the near neutral concentrated feed has a pH of 6.5-8.5.
      Item 32 specifies the method of any one of items 22-32, wherein the alkaline concentrated feed has a pH of 9 or higher, the acidic concentrated feed has a pH of 5 or lower and the near neutral concentrated feed has a pH of 7 to 8.5.
      Item 33 specifies the method of item 32, wherein
   (a) the alkaline concentrated feed has a pH of 9 to 11, the acidic concentrated feed has a pH of 2 to 5 and the near neutral concentrated feed has a pH of 7 to 8.5;
   (b) the alkaline concentrated feed has a pH of 9.8 to 10.8, the acidic concentrated feed has a pH of 3.6 to 4.8 lower and the near neutral concentrated feed has a pH of 7 to 8.5; or
   (c) the alkaline concentrated feed has a pH of 9.8 to 10.5, the acidic concentrated feed has a pH of 3.8 to 4.5 and the near neutral concentrated feed has a pH of 7.5 to 8.5.
      Item 34 specifies the method of item 33, wherein the serum-free cell culture perfusion medium is (a) a chemically defined medium, (b) a hydrolysate-free medium, and/or (c) a protein-free medium or a protein-free medium comprising recombinant insulin and/or recombinant insulin-like growth factor.
      Item 35 specifies the method of any of items 22-34, wherein the separate addition of the three separate concentrated feeds from the diluent enables to control osmolality of the serum-free cell culture perfusion medium in the bioreactor.
      Item 36 specifies the method of any one of items 22-35, wherein
   (a) the ratio (v/v/v) of the alkaline concentrated feed to the acidic concentrated feed to the near neutral concentrated feed is a fixed ratio to provide the serum-free cell culture perfusion medium that is pH-adjusting to a neutral pH in the cell culture and/or the reaction vessel of the bioreactor; and
   (b) the ratio (v/v) of the diluent to the cumulative volume of the at least three separate aqueous concentrated feeds added to the cell culture and/or the reaction vessel of the bioreactor to provide the serum-free cell culture perfusion medium that is pH-adjusting to a near neutral pH determines the osmolality of the serum-free cell culture perfusion medium in the cell culture and/or the reaction vessel of the bioreactor.
      Item 37 specifies the method of any one of items 22-36, wherein the acidic concentrated feed comprises trace elements, trace metals, inorganic salts, chelators, polyamines, and regulatory hormones.
      Item 38 specifies the method of any one of items 22-37, wherein the acidic concentrated feed and/or the near neutral concentrated feed comprise surfactants, anti-oxidants, and carbon sources.
      Item 39 specifies the method of any one of items 22-38, wherein the alkaline concentrated feed comprises amino acids with maximum solubility at alkaline pH of 9 or higher, preferably comprising aspartic acid, histidine, tyrosine, and optionally cysteine and/or cystine and/or folic acid.
      Item 40 specifies the method of item 39 wherein the remaining amino acids are in the acidic and/or near neutral concentrated feed, preferably in the acidic concentrated feed.
      Item 41 specifies the method of any one of items 22-40 wherein the vitamins and the metals are in separate feeds, preferably vitamins are in the near neutral feed and metals are in the acidic feed.
      Item 42 specifies the method of item 41 wherein vitamins poorly soluble in aqueous solutions, such as choline chloride, are present in the neutral feed and the acidic feed.
      Item 43 specifies the method of any one of items 22-42, wherein the cell culture and/or the reaction vessel of the bioreactor comprise at least about 100 L serum-free cell culture perfusion medium, preferably at least about 1000 L serum-free cell culture perfusion medium.
      Item 44 specifies a serum-free cell culture perfusion medium obtainable by the method according to items 22-43.
      Item 45 specifies a method of culturing mammalian cells expressing a heterologous protein in perfusion culture, comprising:
   (a) inoculating a bioreactor with mammalian cells expressing a heterologous protein in a serum-free cell culture medium;
   (b) culturing the mammalian cells in a perfusion culture by continuously feeding the mammalian cells with a serum-free cell culture perfusion medium feed and removing spent media while keeping the cells in culture, wherein the serum-free cell culture perfusion medium feed is (i) a compartmentalized serum-free cell culture perfusion medium comprising the medium components subgrouped into at least three separate aqueous concentrated feeds and a diluent, wherein the first concentrated feed is an alkaline concentrated feed, the second concentrated feed is an acidic concentrated feed and the third concentrated feed is a near neutral concentrated feed; and wherein the compartmentalized serum-free cell culture perfusion medium is pH-adjusting to neutral pH upon mixing of the at least three separate aqueous concentrated feeds and the diluent in the resulting serum-free cell culture perfusion medium; and/or (ii) the serum-free cell culture perfusion medium according to item 44, and
   wherein the alkaline concentrated feed, the acidic concentrated feed and the near neutral concentrated feed of the compartmentalized serum-free cell culture perfusion medium feed are added separately to the cell culture and/or the reaction vessel of the bioreactor and wherein the diluent is added separately to the cell culture and/or the reaction vessel of the bioreactor or the diluent is premixed with one of the at least three separate aqueous concentrated feeds immediately before addition to the cell culture and/or the reaction vessel of the bioreactor.
      Item 46 specifies the method of item 45, wherein the mammalian cells are initially cultured as a batch culture before perfusion culture is started.
      Item 47 specifies the method of items 45 or 46, wherein perfusion culture starts from days 0 to day 3 of the culture.
      Item 48 specifies the method of any one of items 45-47, wherein the perfusion rate increases after perfusion has started until a target viable cell density has been reached.
      Item 49 specifies the method of item 48, wherein the perfusion rate increases from less or equal to 0.5 vessel volumes per day to about 5 vessel volumes per day, or from less or equal to 0.5 vessel volumes per day to about 2 vessel volumes per day.
      Item 50 specifies the method of any one of items 45-49, wherein the osmolality of the serum-free cell culture perfusion medium is increased above the optimal osmolality level for growth, resulting in growth suppression at a target viable cell density, preferably wherein the osmolality level of the serum-free cell culture perfusion medium is increased gradually or stepwise starting at about half the target viable cell density.
      Item 51 specifies the method of any one of items 45-50, wherein the target viable cell density is about 30×10⁶ cells/ml or higher, about 60×10⁶ cells/ml or higher, about 80×10⁶ cells/ml, preferably about 100×10⁶ cells/ml or higher.
      Item 52 specifies the method of any one of items 45-51, wherein the osmolality is controlled using
   (a) a constant concentrated feed perfusion rate and a varying diluent perfusion rate, resulting in a varying overall perfusion rate; or
   (b) a constant overall perfusion rate and a varying concentrated feed perfusion rate;
   wherein the at least three concentrated feeds are added at a fixed ratio (v/v/v) to each other depending on their fold-concentration to maintain the relative proportion of the medium components in the 1× serum-free cell culture perfusion medium.
      Item 53 specifies the method of any one of items 45-52, wherein the osmolality is increased using
   (a) a constant concentrated feed perfusion rate and a decreased diluent perfusion rate, resulting in a decreased overall perfusion rate; or
   (b) a constant overall perfusion rate and an increased concentrated feed perfusion rate with a decreased diluent perfusion rate;
   wherein the at least three concentrated feeds are added at a fixed ratio (v/v/v) to each other depending on their fold-concentration to maintain the relative proportion of the medium components in the 1× serum-free cell culture perfusion medium.
      Item 54 specifies the method of any one of items 50-53, wherein no further additive is added to the culture for increasing the osmolality.
      Item 55 specifies the method of any one of items 50-54, wherein the optimal osmolality level for growth is about 280 to less than 350 mOsm.
      Item 56 specifies the method of any one of items 50-55, wherein the osmolality is maintained at a level optimal for growth until about half the target viable cell density is reached.
      Item 57 specifies the method of any one of items 50-56, wherein the osmolality is increased gradually or stepwise starting at about half the target viable cell density, preferably to about 10-50% of the optimal osmolality level for growth.
      Item 58 specifies the method of any one of items 50-57, wherein the osmolality is increased to and maintained at an osmolality level that suppresses cell growth at about the target viable cell density, wherein the osmolality level that suppresses cell growth is preferably about 350 mOsm or higher, more preferably about 380 mOsm or higher.
      Item 59 specifies the method of any one of items 50-58, wherein increasing the osmolality reduces or eliminates the need for cell bleeding during production phase.
      Item 60 specifies the method of any one of items 50-59, wherein the yield of the heterologous protein produced in the cell culture is increased by at least 5-50% relative to the yield in a control cell culture, wherein the osmolality is not increased.
      Item 61 specifies the method of any one of items 50-60, wherein cell growth is suppressed to maintain a sustainable viable cell density without cell bleeding.
      Item 62 specifies the method of any one of items 45-61, wherein the cell specific perfusion rate (pl/cell/day) is reduced by at least 30% relative to the cell specific perfusion rate of a 1× serum-free cell culture medium.
      Item 63 specifies the method of any one of items 45-62, further comprising harvesting the heterologous protein from the cell culture.
      Item 64 specifies the method of any one of items 45-63, wherein the heterologous protein is a therapeutic protein, an antibody, or a therapeutically effective fragment thereof.
      Item 65 specifies the method of item 64, wherein the antibody is a monoclonal antibody, a bispecific antibody, a multispecific antibody or a fragment thereof.
      Item 66 specifies the method of any one of items 45-65, wherein the mammalian cells comprise Chinese Hamster Ovary (CHO) cells, Jurkat cells, 293 cells, HeLa cells, CV-1 cells, or 3T3 cells, or a derivative of any of these cells, wherein said CHO cell can be further selected from the group consisting of a CHO-DG44 cell, a CHO-K1 cell, a CHO DXB11 cell, a CHO-S cell, and a CHO GS deficient cell or a mutant thereof.
      Item 67 specifies the method of any one of items 45-66, wherein the cell culture and/or the reaction vessel of the bioreactor comprise at least about 100 L serum-free cell culture perfusion medium, preferably at least about 1000 L serum-free cell culture perfusion medium.
      Item 68 specifies the method of any one of items 45-67, wherein further supplements selected from the list of anti-foaming agents, base and glucose are added separately to the cell culture.
      Item 69 specifies a method of producing a therapeutic protein using the method of any one of items 45-68.
      Item 70 specifies a use of the compartmentalized serum-free cell culture perfusion medium of any one of items 1-18 or the serum-free cell culture perfusion medium of item 44 for culturing mammalian cells.
      Item 71 specifies a use of the compartmentalized serum-free cell culture perfusion medium of any one of items 1-18 or the serum-free cell culture perfusion medium of item 44 for culturing mammalian cells in a perfusion culture.
      Item 72 specifies a use of the compartmentalized serum-free cell culture perfusion medium of any one of items 1-18 or the serum-free cell culture perfusion medium of item 44 for controlling osmolality in a perfusion cell culture.
      Item 73 specifies the use of item 72, wherein increasing the osmolality in the cell culture suppresses cell growth and increases heterologous protein production.
      Item 74 specifies the use of item 73, wherein the yield of the heterologous protein produced in the cell culture is increased by at least 5-50% relative to the yield in a control cell culture, wherein the osmolality is not increased.
      Item 75 specifies the use of item 73 or 74, wherein the growth suppression is sufficient to maintain a sustainable viable cell density without cell bleeding.
      Item 76 specifies the use of item 70 or 75, wherein the cell specific perfusion rate (pl/cell/day) is reduced by at least 30% relative to the cell specific perfusion rate of a 1× serum-free cell culture medium.
      Item 77 specifies a use of the compartmentalized serum-free cell culture perfusion medium of any one of items 1-18 for separate addition of the at least three separate aqueous concentrated feeds to a cell culture and/or a reaction vessel of a bioreactor.

### EXAMPLES

### Methods

### Seed Train and Inoculum:

A Chinese Hamster Ovary (CHO) cell line expressing a recombinant IgG was cultured in suspension in Corning-Life Sciences shake flasks (Oneonta, N.Y.) expanded from a 3e7 cell vial in proprietary growth medium. Flasks were seeded at 0.5e6 cells/mL for 3 day passages and 0.8e6 cells/mL for 2 day passages and grown in batch mode, agitated at 120 rpm until the N-3 3 L shake flask which agitated at 80 rpm, at a 50 mm orbital radius. Culture incubators (Infors, Annapolis, Md.) were maintained at 36.5° C., 5% CO2, with no humidity control. The N-2 stages were seeded at 1.0±0.4e6 cells/mL and grown in batch mode for 3 days at 5 L working volume in a GE wave (GE Healthcare). The N-1 stages were run in perfusion mode in a GE Wave 25 system (GE Healthcare). The inoculation densities were 1.0±0.4e6 cells/mL in 25 L working volume. Perfusion was started on day 1 of the culture at 0.5 vessel volume per day (vvd) and ramped up 0.5 vvd each day until 2.0 vvd was reached on day 4, where it remained until day 5 or 6. Run duration was determined based on reaching target viable cell density (VCD): 40e6 c/mL.

### Experimental Bioreactor Set-Up:

The perfusion N-1 culture inoculated 100 L single-use bioreactors (SUB) at a high density of 10±2e6 cells/mL in Boehringer Ingelheims proprietary iSKID (an integrated, continuous bioprocessing system) as disclosed in U.S. provisional application 62/827,504, particularly FIG. 6 thereof. Customized ThermoFisher Hyclone (Logan, Utah) SUB bags were used with the DeltaV distributed control system (Emerson, St Louis, Mo.) to maintain the cultures at 36.5° C., target oxygen set point at 60% air saturation, pH setpoint 7.1, with a single marine impeller operating at 18 W/m³ power per unit volume. A low-shear centrifugal pump (Levitronix, Zurich, Switzerland) was used to recirculate cell culture through a 0.2 um pore-size polyethylene sulfone (PES) tangential flow filtration (TFF) cell retention device (Repligen, Waltham, Mass.) at 13 liters per minute (LPM). The harvest cell culture fluid, or permeate, which passes through the TFF is loaded directly onto the capture columns of the purification unit operation of the iSkid. Growth medium, three concentrated media feeds (acidic, basic, and neutral), 0.1 μm-filtered sterile reverse-osmosis deionized (RODI) water diluent, basic titrant (1M sodium carbonate) to maintain the pH during cultivation, glucose feed (500 g/L), and 1% medical antifoam C emulsion (Dow Corning, Midland, Mich.) were attached to the SUBs via sterile tubing welders or sterile aseptic quick-connectors (Colder Products Company, St Paul, Minn.). All addition lines were separate to avoid precipitation, except in the case of the basic concentrated feed, which was manifolded with the sterile water diluent, followed by an in-line mixer in the tubing before reaching the bioreactor in a single tube.

The perfusion medium (three concentrated media feeds) used has been prepared as follows:

The acidic feed at 1× comprises the following:
the proteinogenic amino acids not found in the basic feed and the non-proteinogenic amino acids hydroxyproline and ornithine to a total of 87.8 mM;
inorganic salts including buffering salts (trace metal salts and iron sources are listed separately) to a total of 21.4 mM;
organic acids taurine and alternative carbon source to a total of 16.3 mM;
combined iron sources to a total of 0.25 mM;
a polyamine at 0.28 mM;
ethanolamine at 0.28 mM;
trace metals (excluding iron) to a total of 0.1 mM;
a first antioxidant at 0.02 m;
vitamins calcium pantothenate at 0.07 mM, thiamine at 0.04 mM, and pyridoxine at 0.3 mM;
choline chloride, separated into the acidic and neutral feeds, is at 1.27 mM in the acidic feed;
a carbon source at 50 mM;
a recombinant protein acting as growth factor at 2.4 μM; and
a surfactant at 0.2 mM.
   These concentrations were increased 6-fold for the 6× concentrated acidic feed used in the examples. The final pH of the 6× concentrated acidic feed is adjusted with sodium hydroxide to 4.2±0.1 and the osmolality 1700±50 mOsm. Although not required the medium has been prepared as a basal powder prior to addition of the carbon source and 1 g/L glucose has been added for milling purposes only.
   The neutral feed at 1× comprises the following:
bicarbonate at 25 mM;
an inorganic buffering salt at 4.1 mM;
inositol at 1.69 mM;

all other vitamins not already included in the acidic feed (but including the remaining choline chloride) to a total of 0.57 mM;

a second antioxidant at 0.01 mM; L-alpha-amino-N-butyric acid at 0.043 mM;

a surfactant at 0.2 mM; and

linoleic acid at 5 μM.
These concentrations were increased 25-fold for the 25× concentrated neutral feed used in the examples. The final pH of the 25× concentrated neutral feed is self-adjusting to 8.0±0.1 without the use of a titrant and the osmolality is 1500±35 mOsm.
   The basic feed at 1× comprises:
the amino acids aspartic acid, histidine, tyrosine, cysteine (including cystine) to a total concentration of 43 mM.
   This concentration was increased 25-fold for the 25× concentrated basic feed used in the examples herewith. The final pH of the 25× concentrated basic feed is adjusted to 10.2±0.1 using sodium hydroxide and has an osmolality 1600±50 mOsm.

The perfusion medium composed of the 3 separate aqueous concentrated feeds, wherein the acidic concentrated feed is a 6× concentrated feed at pH 4.2±0.1 with osmolality of 1700±50 mOsm, the neutral concentrated feed is a 25× concentrated feed at pH 8.0±0.1 with osmolality of 1500±35 mOsm and the basic or alkaline concentrated feed is a 25× concentrated feed at pH 10.2±0.1 with osmolality of 1600±50 mOsm, is pH adjusting to a pH of 7.0±0.1.

### Example 1

After inoculation on day 0, perfusion was started immediately using proprietary growth medium at a rate of 1 vvd. The perfusion rate was increased by 0.5 vvd each day until day 2 when 2.0 vvd was reached. Bioreactor working volume was maintained by controlling media addition via bioreactor weight. On day 2, the concentrated media feeds and diluent replaced the growth medium to start the “production phase,” that is, when the culture reached 0.2 gram/Lbr/day of product in the permeate and loading of the capture columns began. The concentrated feeds were fed at a constant total of 0.5 vvd (acidic feed at 0.33 vvd, basic and neutral feeds at 0.08 vvd each) during the production phase. The rate of feeds was calculated so that the proportions of the nutrients in each feed were kept the same as compared to the intact 1× formulation at 2 vvd using the following equations:

[1x]*2vvd=[6x]*X vvd (eq.1)

where X is the perfusion rate in vvd of the acidic feed necessary to maintain the same nutrient load as in the 1× concentration formulation at 2vvd.

### Similarly,

[1x]*2vvd=[25x]*X vvd (eq.2)

where X is the perfusion rate in vvd of the basic or neutral feed necessary to maintain the same nutrient load as in the 1× concentration formulation at 2vvd.

A VCD maximum of approximately 140±30e6 cells/mL was targeted for the cell line used in these experiments, based on previous engineering runs which showed that range as the maximum sustainable VCD (results not shown). VCD counts were performed on the Beckman CoulterVi-cell (Indianapolis, Ind.). In order to reach this target, which is approximately 15-45% lower than the peak growth capability of this cell line (results not shown), the osmolality of the culture was gradually increased to inhibit cell replication. Culture osmolality was measured with the BioProfile FLEX analyzer (Nova Biomedical, Waltham, Mass.), all other culture metabolites were measured with the Roche Cedex BioAnalyzer (Indianapolis, Ind.). Osmolality increase was achieved by adjusting the diluent rate daily to reach the target residual osmolality of the culture while the rate of feeds addition remained constant. Thus, the overall perfusion rate varied from day to day. An osmo balance for the daily osmolality consumption was calculated according to the following equation:

osmo input−osmo output=osmo consumption (eq. 3)

where osmo input is the osmolality of media concentrate feeds and diluent perfusing into the bioreactor, osmo output is the residual osmolality of the bioreactor supernatant, and osmo consumption is the difference in osmolality between the input and output. This daily osmo consumption is then normalized to the number of cells in the culture, for a daily per cell osmolality consumption. This daily consumption rate per cell (or cell-specific osmo consumption rate, CSOCR) was then multiplied by the following day's predicted VCD to predict the following day's osmo consumption. This consumption rate was then used in eq 3, along with the desired osmo output, to calculate the required osmo input for the following day. The perfusion rate of the diluent was therefore adjusted to match the osmo input target while the feeds were maintained. The desired osmo target and approximate perfusion rate for each day varied according to Table 1 (values varied from run to run, resulting in the following ranges):

| | Approximate | Target | Approximate |
|---|---|---|---|
| | Viable cell density | osmolality | Perfusion rate |
| Day | (e6 c/mL) | (mOsm) | (VVD) |
| 2 | 25 | 300-330 | 1.6-1.8 |
| 3 | 50 | 300-330 | 2 |
| 4 | 75 | 330-360 | 1.8-2 |
| 5 | 100 | 350-380 | 1.5-1.6 |
| 6 | 130-150 | 380-410 | 1.2-1.4 |
| 7 | 150-170 | 380-410 | 1.2-1.7 |
| 8 | 150-170 | 380-410 | 1.2-1.3 |
| 9 | 140-170 | 380-410 | 1.2-1.3 |
| 10 | 140-180 | 380-410 | 1.2-1.3 |
| 11 | 130-180 | 380-410 | 1.2-1.3 |
| 12 | 130-170 | 380-410 | 1.2-1.3 |
| 13 | 130-170 | 380-410 | 1.2-1.4 |
| 14 | 120-160 | 380-410 | 1.3-1.4 |

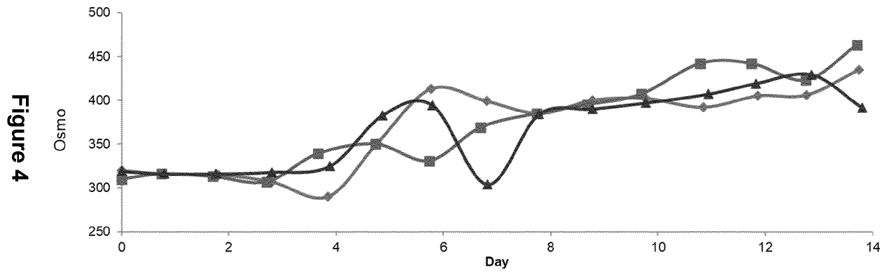

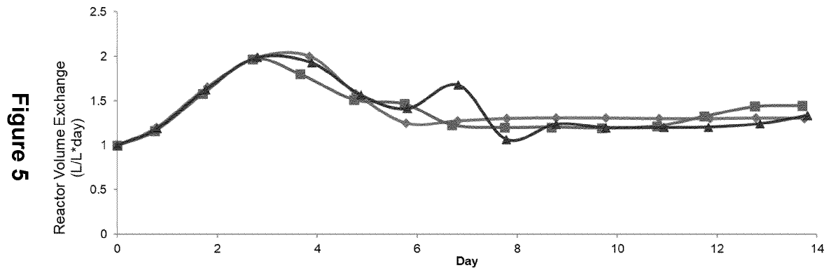

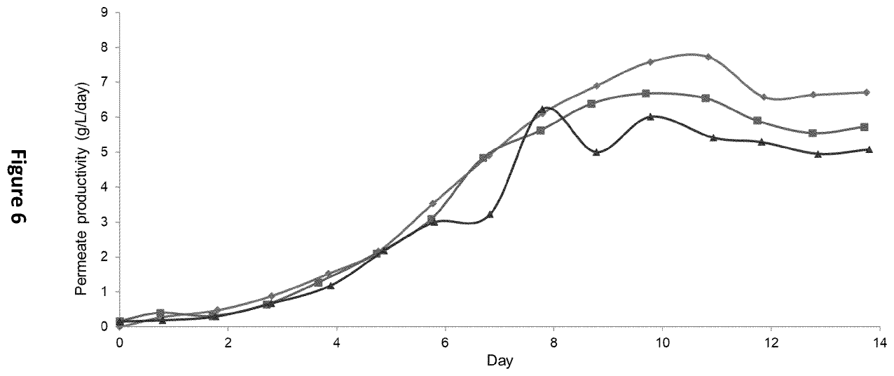

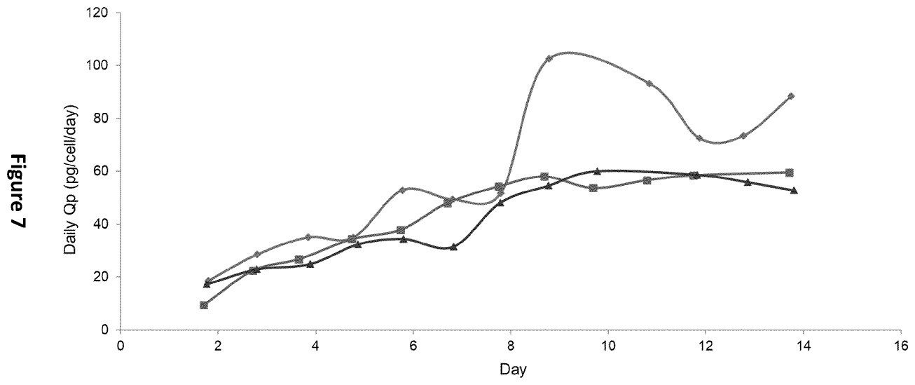

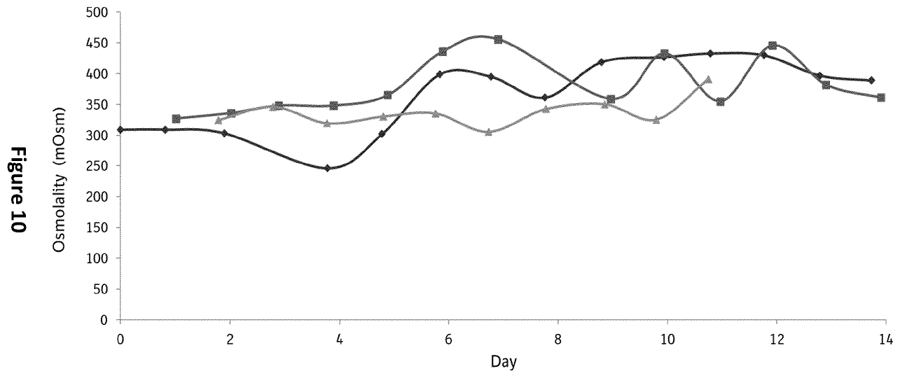

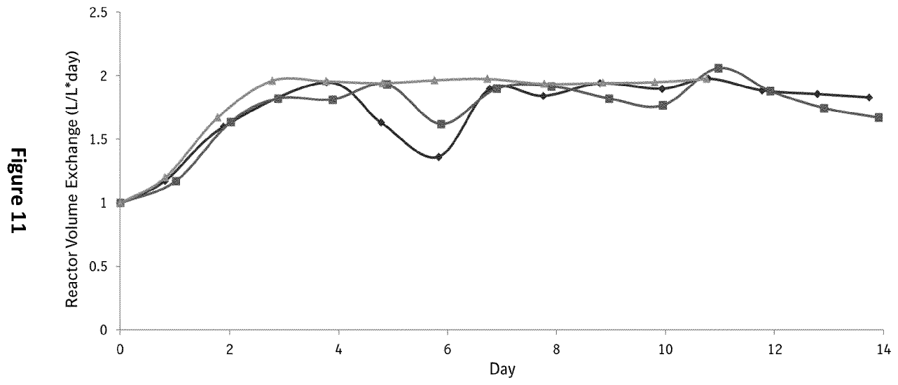

Daily glucose measurements were taken and separate glucose bolus feeds were added as necessary to maintain residual glucose at or above 2 g/L. Cultures were terminated at 14 days based on a business case for matching the run duration of a typical fed-batch culture. The results of the three 100 L bioreactor runs are shown in FIG. 3 (VCD), FIG. 4 (Osmolality), FIG. 5 (reactor volume exchange), FIG. 6 (permeate productivity), FIG. 7 (daily specific productivity) and FIG. 8 (cell specific perfusion rate).

### Example 2

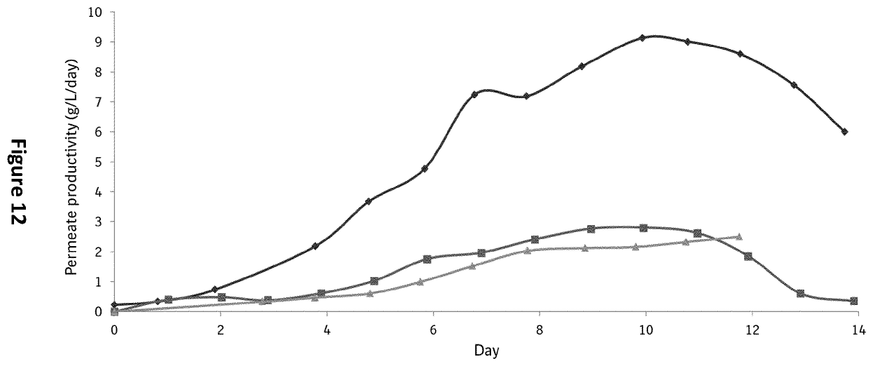

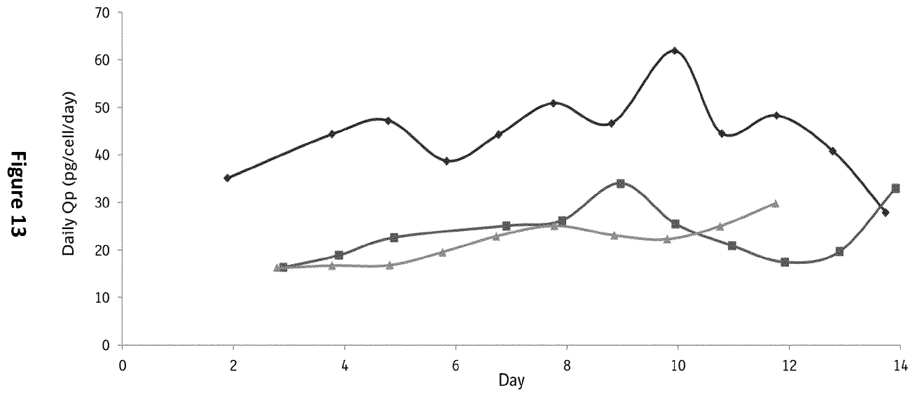

Three CHO cell lines A (⋄), B (□), and C (Δ) (see FIGS. 9 to 14) expressing different recombinant IgG molecules were cultured in a 2 L bioreactor. After inoculation on day 0, perfusion was started immediately using proprietary growth medium at a rate of 1 vvd. The perfusion rate was increased by 0.5 vvd each day until day 2 when 2.0 vvd was reached. Bioreactor working volume was maintained by controlling media addition via bioreactor weight. On day 2, the concentrated media feeds and diluent replaced the growth medium to start the “production phase,” that is, when the culture reached 0.2 gram/Lbr/day of product in the permeate and loading of the capture columns began. The cells were fed with a constant volume of about 2 vvd with varying proportions of the three concentrated media feeds and sterile water diluents. The rate of feeds was calculated so that the proportions of the nutrients in each feed were kept the same as compared to the intact 1× formulation at 2 vvd as explained in Example 1.

A VCD maximum of approximately 180±30e6 cells/mL and 140±30e6 cells/mL were targeted for cell lines A and B, respectively, based on previous engineering runs which showed that range as the maximum sustainable VCD for these cell lines (results not shown). Cell line C had a maximum peak VCD of 100±20e6 c/mL, therefore no suppression of growth was necessary for that cell line and osmolality was maintained within the physiologically optimum range of 330±30 mOsm. VCD counts were performed on the Beckman Coulter Vi-cell (Indianapolis, Ind.). In order to reach targets for cell lines A and B, which are approximately 15-45% lower than the peak growth capabilities of these cell lines (results not shown), the osmolality of the cultures was gradually increased to inhibit cell replication. Culture osmolality was measured with the BioProfile FLEX analyzer (Nova Biomedical, Waltham, Mass.), all other culture metabolites were measured with the Roche Cedex BioAnalyzer (Indianapolis, Ind.). Osmolality increase was achieved by adjusting the concentrated feed rate and the diluent rate daily to reach the target residual osmolality of the culture while the total VVD addition remained constant at two vvd. The daily osmolality consumption was calculated as explained in Example 1 according to the following equation:

Osmo input−osmo output+osmo consumption.

As in example 1, the daily osmo consumption rate was determined and then used to calculate the osmo input necessary to achieve the new desired osmo output for the following day. However, in the case for example 2 osmo control strategy, the rates for both the feeds and diluent are adjusted (as opposed to diluent alone in example 1) to achieve the target osmo input at an overall perfusion rate of 2 vvd.

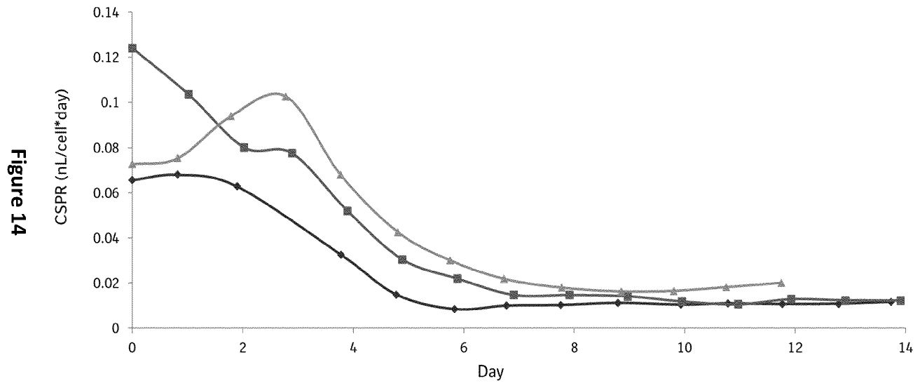

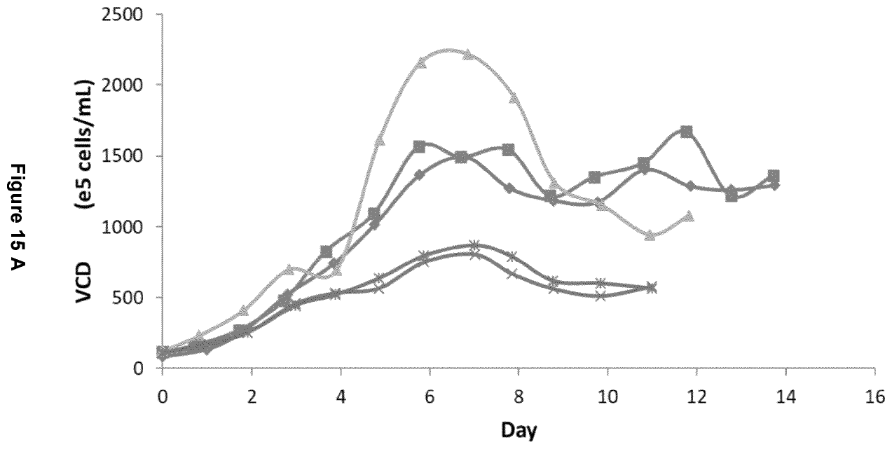

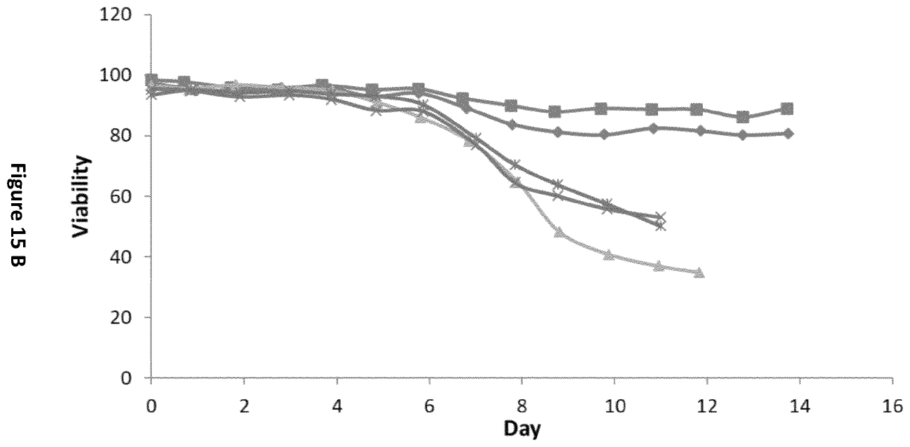

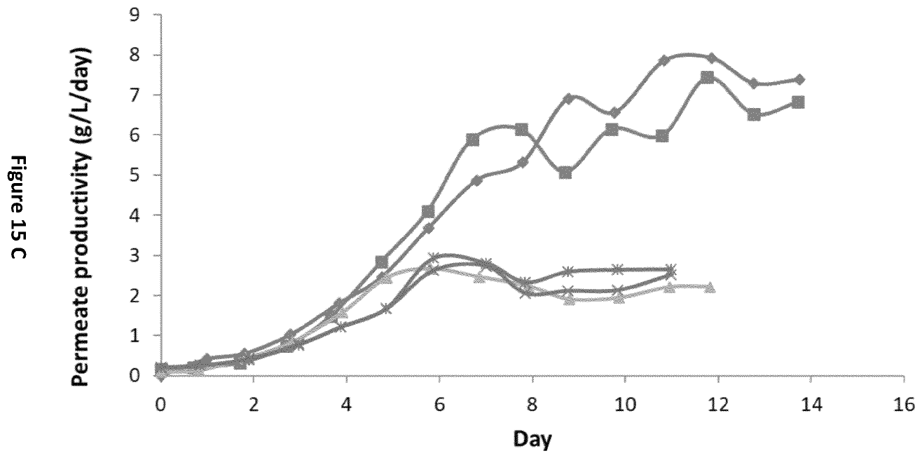

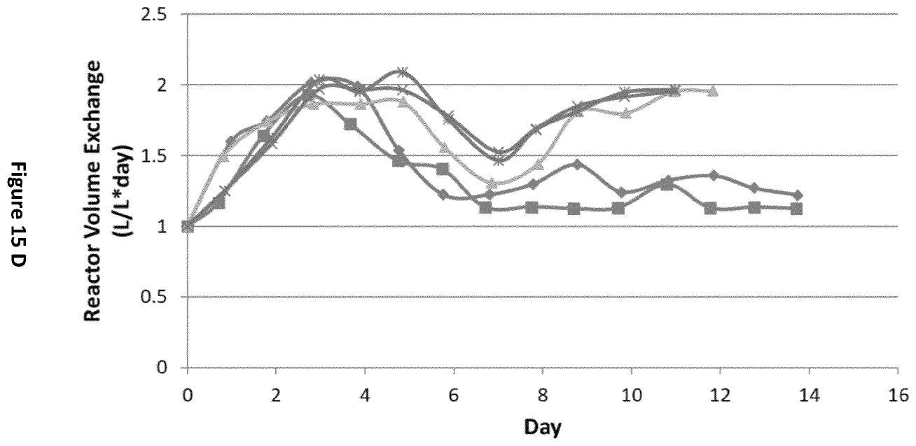

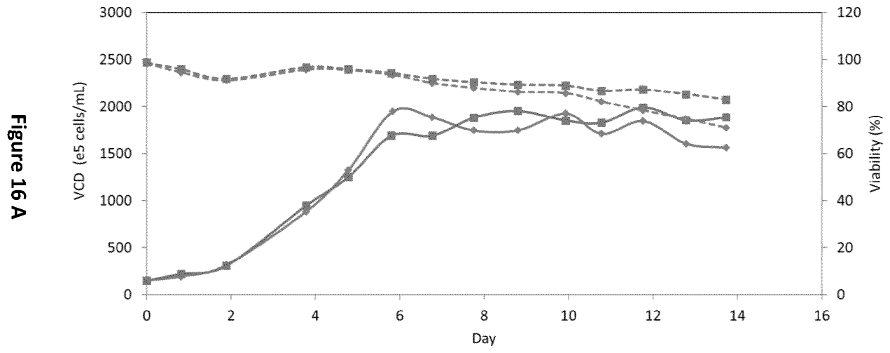

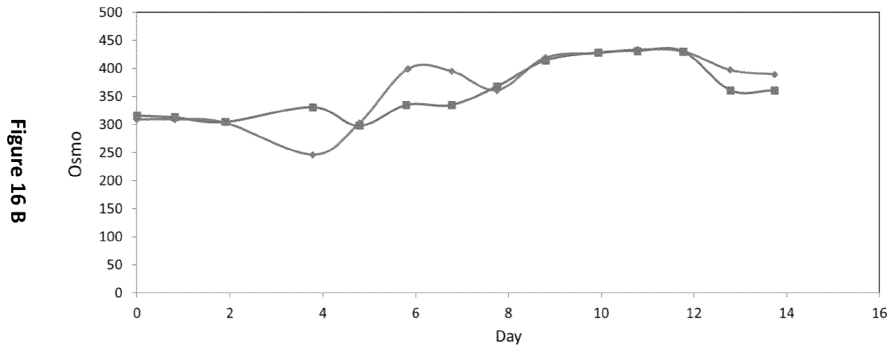

Daily glucose measurements were taken and separate glucose bolus feeds were added as necessary to maintain residual glucose at or above 2 g/L. Cultures were terminated at 14 days based on a business case for matching the run duration of a typical fed-batch culture, without the need for cell bleeding. The results of the three 2 L bioreactor runs are shown in FIG. 9 (VCD), FIG. 10 (Osmolality), FIG. 11 (reactor volume exchange), FIG. 12 (permeate productivity), FIG. 13 (daily specific productivity) and FIG. 14 (cell specific perfusion rate).

### Example 3

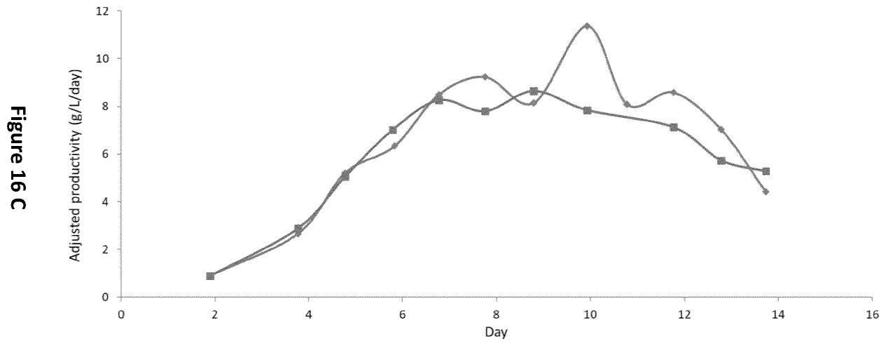

A CHO DG44 cell line expressed in the dihydrofolate reductase (dhfr) selection system (cell line A, Δ) and two different CHO-K1 cell lines run in duplicates (cell line B □, ⋄; cell line C x, x) expressed in the glutamine synthetase (GS) selection system (see FIG. 15) were cultured in a 2 L bioreactor using three concentrated media feeds fixed at a total of 0.5 vessel volumes per day (VVD) with varying diluent volume. All cell lines express a different recombinant IgG molecule. Bioreactor working volume was maintained by controlling media addition via bioreactor weight. On day 2, the concentrated media feeds and diluent replaced the growth medium to start the “production phase,” that is, when the culture reached 0.2 gram/L_{bioreactor}/day of product in the permeate and loading of the capture columns began. The concentrated feeds were fed at a constant total of 0.5 vvd (acidic feed at 0.33 vvd, basic and neutral feeds at 0.08 vvd each) during the production phase. The rate of feeds was calculated so that the proportions of the nutrients in each feed were kept the same as compared to the intact 1× formulation at 2 vvd as explained in Example 1.

Cell line A was cultured at physiologically optimum osmolality (330±30 mOsm) for the entre culture duration (12 days) to promote maximum cell culture growth (ie peak possible VCD). This is considered the “engineering” or development run for this cell line. Cell line B targeted a VCD maximum of 150±30e6 cells/mL±20e6 c/mL, based on such a previous engineering run which showed that range as the maximum sustainable VCD for this cell lines (results not shown). Cell line C had a maximum peak VCD of <100±20e6 c/mL, therefore no suppression of growth was necessary for that cell line and osmolality was maintained within the physiologically optimum range of 330±30 mOsm. VCD counts were performed on the Beckman Coulter Vi-cell (Indianapolis, Ind.). In order to reach target for cell line B, which was approximately 15-45% lower than the peak growth capabilities of this cell line (results not shown), the osmolality of the culture was gradually increased to inhibit cell replication. Culture osmolality was measured with the BioProfile FLEX analyzer (Nova Biomedical, Waltham, Mass.), all other culture metabolites were measured with the Roche Cedex BioAnalyzer (Indianapolis, Ind.). Osmolality increase was achieved by adjusting the diluent rate daily to reach the target residual osmolality of the culture while the rate of feeds addition remained constant. The daily osmolality consumption was calculated as explained in Example 1 according to the following equation:

Osmo input−osmo output+osmo consumption

As in example 1, the daily osmo consumption rate was determined and then used to calculate the osmo input necessary to achieve the new desired osmo output for the following day.

Daily glucose measurements were taken and separate glucose bolus feeds were added as necessary to maintain residual glucose at or above 2 g/L. Cultures were terminated at 11, 12 and 14 days as shown in FIG. 15. The results of the 2 L bioreactor runs are shown in FIG. 15A viable cell densities (VCD; e5 c/mL); FIG. 15B viability (%); FIG. 15C permeate productivity (g/L/day), with the permeate productivity being calculated from the daily instantaneous titer of the permeate (g/L_{media}), as measured by the Cedex BioAnalyzer, multiplied by the daily perfusion rate (L_{media}/L_{bioreactor}/day); and FIG. 15D perfusion rate expressed in reactor volume exchange (L_{media}/L_{bioreactor}/day).

### Example 4

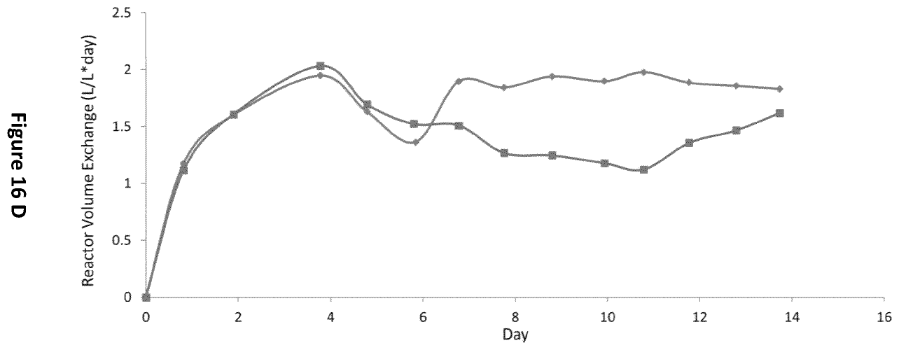

A CHO-K1 cell line expressing a recombinant IgG in the glutamine synthetase (GS) selection system was cultured in a 2 L bioreactor. Runs were performed in either the “MCs vary, total VVD fixed” (⋄) or “MCs fixed, total VVD vary” (□) perfusion control modes as described in Examples 2 and 3. “MCs vary, total VVD fixed” refers to a constant total vessel volume per day (VVD) perfusion rate achieved by varying the perfusion rate of the combined Media Concentrates (MCs) and concomitantly varying diluent rate to maintain 2 VVD. “MCs fixed, total VVD vary” refers to a constant perfusion rate of MCs at 0.5 VVD with a varying diluent perfusion rate, for an overall fluctuating perfusion rate. Both perfusion control modes are capable of manipulating media osmolality to set targets (FIG. 16 B). Viability and viable cell density are comparable using the two perfusion control modes for this cell line. The separation of media concentrate feeds (ie the nutrient delivery) from the diluent enables a low perfusion rate (2 VVD) with the ability to supply adequate nutrients at high cell density by changing the proportion of media concentrates to diluent. Residual culture osmolality can thus be controlled to elevated levels which are higher than the physiologically optimal range (varies depending on cell line; for this cell line 300-330 mOsm) without increasing the perfusion rate beyond 2 VVD (the highest perfusion rate considered scalable to >100 L bioreactor by this company). When the osmolality is increased before the peak viable cell density (VCD) is reached, the peak VCD can be suppressed (see FIGS. 3 and 4).

Adjusted productivity (FIG. 16C) was determined as the total productivity for the system, i.e. including the product in the permeate and retained within the bioreactor each day. The productivity of the two perfusion control modes is similar for the cell line shown, therefor either perfusion mode may be selected as the process for this cell line. Reactor volume exchange (L_{media}/L_{bioreactor}/day), or perfusion rate, for cell cultures are shown in FIG. 16D. Due to operator error on days 4 and 5 of run “MCs vary, total VVD fixed” the target 2 VVD was not reached those days. All remaining days of the production phase (>day 2) were maintained at target 2 VVD. “MCs fixed, total VVD vary” run shows the variable perfusion rate which was necessary to maintain target osmolality (FIG. 16b).

1. A compartmentalized serum-free cell culture perfusion medium comprising the medium components subgrouped into at least three separate aqueous concentrated feeds and a diluent, wherein the first concentrated feed is an alkaline concentrated feed, the second concentrated feed is an acidic concentrated feed and the third concentrated feed is a near neutral concentrated feed; wherein
the compartmentalized serum-free cell culture perfusion medium is automatically adjusted to neutral pH upon mixing of the at least three separate aqueous concentrated feeds and the diluent in the resulting serum-free cell culture perfusion medium.

2. The compartmentalized serum-free cell culture perfusion medium of claim 1, wherein the resulting serum-free cell culture perfusion medium has a pH of between 6.7 and 7.5 upon mixing of the at least three separate aqueous concentrated feeds and the diluent.

3. The compartmentalized serum-free cell culture perfusion medium of claim 1, wherein the diluent is sterile water.

4. The compartmentalized serum-free cell culture perfusion medium of claim 1, wherein the compartmentalized serum-free cell culture perfusion medium is for
(a) separate addition of the alkaline concentrated feed, the acidic concentrated feed and the near neutral concentrated feed to a cell culture and/or a reaction vessel of a bioreactor;
(b) direct addition of the alkaline concentrated feed, the acidic concentrated feed and the near neutral concentrated feed to a cell culture and/or a reaction vessel of a bioreactor without prior pre-mixing; and/or
(c) direct mixing of the at least three separate aqueous concentrated feeds in a cell culture and/or a reaction vessel of a bioreactor.

5. The compartmentalized serum-free cell culture perfusion medium of claim 1, wherein the alkaline concentrated feed is a 2× to 80× concentrated feed, the acidic concentrated feed is a 2× to 40× concentrated feed and the near neutral concentrated feed is a 2× to 50× concentrated feed.

6. The compartmentalized serum-free cell culture perfusion medium of claim 1, wherein the alkaline concentrated feed has a pH of 9 or higher, the acidic concentrated feed has a pH of 5 or lower and the near neutral concentrated feed has a pH of 7 to 8.5.

7. A first aqueous concentrated feed for combination with a second aqueous concentrated feed, a third aqueous concentrated feed and a diluent to form a serum-free cell culture perfusion medium, wherein the pH of the resulting serum-free cell culture perfusion medium is automatically adjusted to a neutral pH; wherein
(a) the first aqueous concentrated feed is an alkaline aqueous concentrated feed, the second aqueous concentrated feed is an acidic aqueous concentrated feed, and the third aqueous concentrated feed is a near neutral aqueous concentrated feed;
(b) the first aqueous concentrated feed is an acidic aqueous concentrated feed, the second aqueous concentrated feed is an alkaline aqueous concentrated feed, and the third aqueous concentrated feed is a near neutral aqueous concentrated feed; or
(c) the first aqueous concentrated feed is a near neutral aqueous concentrated feed, the second aqueous concentrated feed is an acidic aqueous concentrated feed, and the third aqueous concentrated feed is an alkaline aqueous concentrated feed.

8-9. (canceled)

10. A method of preparing a serum-free cell culture perfusion medium comprising
(a) providing the components of a cell culture media in at least three subgroups of components based on solubility at alkaline, acidic and neutral pH,
(b) dissolving
(i) the subgroup of components soluble at alkaline pH in an alkaline aqueous solution to form an alkaline concentrated feed;
(ii) the subgroup of components soluble at acidic pH in an acidic aqueous solution to form an acidic concentrated feed; and
(iii) the subgroup of components soluble at neutral pH in a neutral aqueous solution to form a near neutral concentrated feed;
(c) optionally storing the prepared alkaline concentrated feed, acidic concentrated feed and near neutral concentrated feed in separate containers; and
(d) adding the prepared alkaline concentrated feed, acidic concentrated feed and near neutral concentrated feed and the diluent to a cell culture and/or the reaction vessel of a bioreactor, wherein
(i) the alkaline concentrated feed, the acidic concentrated feed and the near neutral concentrated feed are added separately to the cell culture and/or the reaction vessel of the bioreactor; and
(ii) the diluent is added separately to the cell culture and/or the reaction vessel of the bioreactor or the diluent is premixed with one of the at least three separate aqueous concentrated feeds immediately before addition to the cell culture and/or the reaction vessel of the bioreactor;
wherein the pH of the resulting serum-free cell culture perfusion medium is automatically pH adjusted to a neutral pH upon mixing of the at least three separate aqueous concentrated feeds and the diluent.

11. A serum-free cell culture perfusion medium obtainable by the method according to claim 10.

12. A method of culturing mammalian cells expressing a heterologous protein in perfusion culture, comprising:
(a) inoculating a bioreactor with mammalian cells expressing a heterologous protein in a serum-free cell culture medium;
(b) culturing the mammalian cells in a perfusion culture by continuously feeding the mammalian cells with a serum-free cell culture perfusion medium feed and removing spent media while keeping the cells in culture, wherein the serum-free cell culture perfusion medium feed is (i) a compartmentalized serum-free cell culture perfusion medium comprising the medium components subgrouped into at least three separate aqueous concentrated feeds and a diluent, wherein the first concentrated feed is an alkaline concentrated feed, the second concentrated feed is an acidic concentrated feed and the third concentrated feed is a near neutral concentrated feed; and wherein the compartmentalized serum-free cell culture perfusion medium is automatically adjusted to neutral pH upon mixing of the at least three separate aqueous concentrated feeds and the diluent in the resulting serum-free cell culture perfusion medium; and/or (ii) the serum-free cell culture perfusion medium according to claim 11, and
wherein the alkaline concentrated feed, the acidic concentrated feed and the near neutral concentrated feed of the compartmentalized serum-free cell culture perfusion medium feed are added separately to the cell culture and/or the reaction vessel of the bioreactor and wherein the diluent is added separately to the cell culture and/or the reaction vessel of the bioreactor or the diluent is premixed with one of the at least three separate aqueous concentrated feeds immediately before addition to the cell culture and/or the reaction vessel of the bioreactor.

13. A method of producing a therapeutic protein using the method of claim 12.

14. A method for culturing mammalian cells comprising culturing the cells in the compartmentalized serum-free cell culture perfusion medium of claim 1.

15. A method for culturing mammalian cells in a perfusion culture comprising culturing, in a perfusion culture, the cells in the compartmentalized serum-free cell culture perfusion medium of claim 1.

16. A method for controlling osmolality in a perfusion culture, said method comprising adding to the perfusion culture the compartmentalized serum-free cell culture perfusion medium of claim 1.

17. A method comprising separately adding the at least three separate aqueous concentrated feeds according to claim 1 to a cell culture and/or a reaction vessel of a bioreactor.

18. A method for culturing mammalian cells comprising culturing the cells in the serum-free cell culture perfusion medium of claim 11.

19. A method for culturing mammalian cells in a perfusion culture comprising culturing, in a perfusion culture, the cells in the serum-free cell culture perfusion medium of claim 11.

20. A method for controlling osmolality in a perfusion culture, said method comprising adding to the perfusion culture the serum-free cell culture perfusion medium of claim 11.
